(19) United States
(12) Patent Application Publication
(10) Pub. No.: US 2020/0205406 A1
(43) Pub. Date: Jul. 2, 2020

(54) MICROBIOCIDAL PHENYLAMIDINE DERIVATIVES WITH IMPROVED PLANT SAFETY PROPERTIES

(71) Applicant: SYNGENTA PARTICIPATIONS AG, Basel (CH)

(72) Inventors: Matthias WEISS, Stein (CH); Ulrich Johannes HAAS, Stein (CH); Clemons LAMBERTH, Stein (CH)

(73) Assignee: SYNGENTA PARTICIPATIONS AG, Basel (CH)

(21) Appl. No.: 16/499,424

(22) PCT Filed: Mar. 27, 2018
(86) PCT No.: PCT/EP2018/057736

§ 371 Date: Sep. 30, 2019

(30) Foreign Application Priority Data
Mar. 31, 2017 (EP) 17164105.3

(51) Int. Cl. A01N 35/10; A01N 43/78; C07C 251/08; C07D 277/34

(52) U.S. Cl. CPC A01N 35/10; C07D 277/34; C07C 251/08; A01N 43/78

(57) ABSTRACT

A method of improving plant safety and/or reducing phytotoxicity and/or reducing plant necrosis whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I) wherein R¹, R₂, R³, R⁴ and R⁵ are as defined in claim 1. Furthermore, the present invention relates to novel compounds of formula (I) and to agrochemical compositions which comprise them, to the preparation of these compounds and compositions, and to the use of the compounds or compositions in the aforementioned methods.

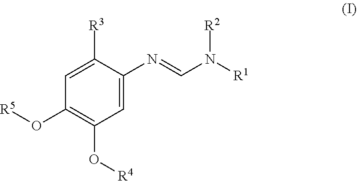

The present invention relates to phenylamidine derivatives, to processes for preparing them, to agrochemical compositions comprising them and to methods of using them in agriculture or horticulture for controlling or preventing infestation of plants, harvested food crops, seeds or non-living materials by phytopathogenic microorganisms, in particular phytopathogenic fungi.

Certain fungicidal phenylamidine derivatives are described in WO 2000/046184, WO07031513, WO 2008/110313 and WO 2010/086118. However, their use as agrochemical fungicides may be limited because of well-known crop-damaging phytotoxic effects of these compounds (i.e. they suffer from a poor plant-safety profile). The phytotoxic effects resulting from phenylamidine derivatives can be seen for example in WO 2008/110278, where the use of these compounds as herbicidal agents is disclosed.

There exists therefore a need for the development of compounds and methods for improving plant safety whilst combating, preventing or controlling phytopathogenic diseases in plants.

It has now surprisingly been found that certain phenylamidine derivatives display favourable plant safety properties whilst controlling phytopathogenic microorganisms, in particular phytopathogenic fungi.

The present invention therefore provides a method of improving plant safety whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I)

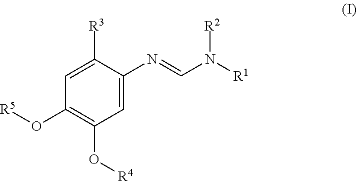

wherein,
R¹ and R² are each independently selected from C₁-C₄alkyl and C₃-C₈cycloalkyl; or
R¹ and R² together with the nitrogen atom to which they are attached form a three to six-membered saturated cyclic group which may optionally contain one oxygen or one sulphur atom;
R³ is C₁-C₄ alkyl or halogen;
R⁴ is C₁-C₄alkyl or C₁-C₄haloalkyl;
R⁵ is aryl (optionally substituted with one to three R⁶ groups) or heteroaryl (optionally substituted with one to three R⁶ groups); and
each R⁶ is independently selected from halogen, cyano, hydroxyl, amino, nitro, C₁-C₄alkyl, C₁-C₄haloalkyl, C₃-C₆cycloalkyl, C₃-C₆halocycloalkyl, C₁-C₄alkoxy, C₁-C₄haloalkoxy, C₃-C₆cycloalkoxy, C₁-C₄alkylthio, C₁-C₄haloalkylthio, C₃-C₆cycloalkylthio, C₁-C₄alkylsulfinyl, C₁-C₄haloalkylsulfinyl, C₁-C₄alkylsulfonyl, C₁-C₄haloalkylsulfonyl, C₁-C₄alkylcarbonyl, C₁-C₄alkoxycarbonyl, C₁-C₄alkylcarbonyloxy, C₂-C₆alkenyl, C₂-C₆haloalkenyl, C₂-C₆alkenyloxy, C₂-C₆haloalkenyloxy, C₂-C₆alkynyl, C₃-C₆cycloalkylC₂-C₆alkynyl, C₂-C₆alkynyloxy, aryl, aryl(C₁-C₄)alkyl, aryloxy, heteroaryl, heteroaryl(C₁-C₄)alkyl and heteroaryloxy; or a salt or an N-oxide thereof.

Surprisingly, compounds of formula (I) are able to control phytopathogenic fungi whilst in addition displaying reduced phytotoxicity on plants, in particular soybean plants.

Thus, in a second aspect of the invention there is provided a method of reducing phytotoxicity whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), as defined above, or a salt or an N-oxide thereof.

In particular, the compounds of formula (I) when used as agrochemical fungicides exhibit reduced plant necrosis.

Thus, in a further aspect of the invention there is provided a method of reducing plant necrosis whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), as defined above, or a salt or an N-oxide thereof.

In a yet further aspect the present invention provides the use of a compound of formula (I) or a salt or N-oxide thereof, or a composition comprising a compound of formula (I) or a salt or N-oxide thereof, as described herein to improve plant safety and/or reduce phytotoxicity and/or reduce plant necrosis whilst controlling phytopathogenic fungi.

The present further relates to novel compounds of formula (I), and salts and N-oxides thereof, which are useful in the aforementioned methods and/or which possess enhanced properties as agrochemical fungicides, and to compositions comprising such compounds and salts and N-oxides thereof.

Thus, in a yet further aspect the present invention provides novel compositions comprising a compound of formula (I), and use of such compounds in the aforementioned methods.

Where substituents are indicated as being optionally substituted, this means that they may or may not carry one or more identical or different substituents, e.g. one to three substituents. Normally not more than three such optional substituents are present at the same time. Where a group is indicated as being substituted, e.g. alkyl, this includes those groups that are part of other groups, e.g. the alkyl in alkylthio.

The term “halogen” refers to fluorine, chlorine, bromine or iodine, preferably fluorine, chlorine or bromine.

Alkyl substituents may be straight-chained or branched. Alkyl on its own or as part of another substituent is, depending upon the number of carbon atoms mentioned, for example, methyl, ethyl, n-propyl, n-butyl, n-pentyl, n-hexyl and the isomers thereof, for example, iso-propyl, iso-butyl, sec-butyl, tert-butyl or iso-amyl.

Alkenyl substituents (either alone or as part of a larger group, eg. alkenyloxy) can be in the form of straight or branched chains, and the alkenyl moieties, where appropriate, can be of either the (E)- or (Z)-configuration. Examples are vinyl and allyl. The alkenyl groups are preferably C₂-C₆, more preferably C₂-C₄ and most preferably C₂-C₃ alkenyl groups.

Alkynyl substituents (either alone or as part of a larger group, eg. alkynyloxy) can be in the form of straight or branched chains. Examples are ethynyl and propargyl. The alkynyl groups are preferably C₂-C₆, more preferably C₂-C₄ and most preferably C₂-C₃ alkynyl groups.

Haloalkyl groups (either alone or as part of a larger group, eg. haloalkyloxy) may contain one or more identical or different halogen atoms and, for example, may stand for CH₂Cl, CHCl₂, CCl₃, CH₂F, CHF₂, CF₃, CF₂Cl, CF₃CH₂, CH₃CF₂, CF₃CF₂ or CCl₃CCl₂.

Haloalkenyl groups (either alone or as part of a larger group, eg. haloalkenyloxy) are alkenyl groups, respectively, which are substituted with one or more of the same or different halogen atoms and are, for example, 2,2-difluorovinyl or 1,2-dichloro-2-fluoro-vinyl.

Alkoxy means a radical —OR, where R is alkyl, e.g. as defined above. Alkoxy groups include, but are not limited to, methoxy, ethoxy, 1-methylethoxy, propoxy, butoxy, 1-methylpropoxy and 2-methylpropoxy.

Cyano means a —CN group.

Nitro means a —NO₂ group.

Amino means an —NH₂ group.

Hydroxyl or hydroxy stands for a —OH group.

Cycloalkyl may be saturated or partially unsaturated, preferably fully saturated, and is, for example, cyclopropyl, cyclobutyl, cyclopentyl, cyclohexyl, or cyclohexenyl.

Aryl groups (either alone or as part of a larger group, such as e.g. aryloxy, aryl-alkyl) are aromatic ring systems which can be in mono-, bi- or tricyclic form. Examples of such rings include phenyl, naphthyl, anthracenyl, indenyl or phenanthrenyl. Preferred aryl groups are phenyl and naphthyl, phenyl being most preferred. Where an aryl moiety is said to be substituted, the aryl moiety is preferably substituted by one to four substituents, most preferably by one to three substituents.

Heteroaryl groups (either alone or as part of a larger group, such as e.g. heteroaryloxy, heteroaryl-alkyl) are aromatic ring systems containing at least one heteroatom and consisting either of a single ring or of two or more fused rings. Preferably, single rings will contain up to three heteroatoms and bicyclic systems up to four heteroatoms which will preferably be chosen from nitrogen, oxygen and sulfur. Examples of monocyclic groups include pyridyl, pyridazinyl, pyrimidinyl, pyrazinyl, pyrrolyl, pyrazolyl, imidazolyl, triazolyl (e.g. [1,2,4] triazolyl), furanyl, thiophenyl, oxazolyl, isoxazolyl, oxadiazolyl, thiazolyl, isothiazolyl and thiadiazolyl. Examples of bicyclic groups include purinyl, quinolinyl, cinnolinyl, quinoxalinyl, indolyl, indazolyl, benzimidazolyl, benzothiophenyl and benzothiazolyl. Monocyclic heteroaryl groups are preferred, pyridyl being most preferred. Where a heteroaryl moiety is said to be substituted, the heteroaryl moiety is preferably substituted by one to four substituents, most preferably by one to three substituents.

The presence of one or more possible asymmetric carbon atoms in a compound of formula (I) means that the compounds may occur in optically isomeric forms, i.e. enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers. The present invention includes all possible tautomeric forms fora compound of formula (I).

In each case, the compounds of formula (I) are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

Preferred values of R¹, R², R³, R⁴, R⁵ and R⁶ are, in any combination thereof, as set out below:

Preferably R¹ and R² are each independently C₁-C₄ alkyl.

More preferably R¹ and R² are each independently selected from methyl, ethyl and isopropyl.

Even more preferably R¹ and R² are each independently selected from methyl and ethyl.

Most preferably R¹ is methyl and R² is ethyl.

Preferably R³ is C₁-C₃ alkyl.

More preferably R³ is methyl, ethyl or isopropyl.

Even more preferably R³ is methyl or ethyl.

Most preferably R³ is methyl.

Preferably R⁴ is C₁-C₄alkyl.

More preferably R⁴ is C₁-C₃alkyl.

Even more preferably R⁴ is methyl or ethyl.

Most preferably R⁴ is methyl.

It is most preferred if both of R³ and R⁴ are methyl.

Preferably R⁵ is phenyl (optionally substituted with one to three R⁶ groups), pyridyl (optionally substituted with one to three R⁶ groups) or thiazolyl (optionally substituted with one to three R⁶ groups);

More preferably R⁵ is phenyl (optionally substituted with one or two R⁶ groups) or thiazolyl (optionally substituted with one or two R⁶ groups).

Even more preferably R⁵ is phenyl (optionally substituted with one or two R⁶ groups);

Most preferably R⁵ is phenyl, which is optionally substituted by one or two substituents independently selected from trifluoromethyl and halogen (preferably fluoro or chloro).

Preferably each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₃-C₆cycloalkoxy, C₁-C₄alkylthio, C₁-C₄alkylsulfonyl, C₁-C₄haloalkylsulfonyl, C₁-C₄alkylcarbonyl, C₂-C₆alkenyl, C₂-C₆haloalkenyl, C₂-C₆alkynyl, phenyl, benzyl, phenoxy, pyridyl, pyridylmethyl and pyridyloxy.

More preferably each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₁-C₄alkylthio, C₁-C₄alkylsulfonyl, C₁-C₄alkylcarbonyl, C₂-C₆alkynyl, phenyl, phenoxy and pyridyl.

Even more preferably each R⁶ is independently selected from halogen, cyano, C₁-C₄alkyl, C₁-C₄haloalkyl, C₁-C₄alkoxy, C₁-C₄haloalkoxy, C₂-C₆alkynyl and phenyl.

Most preferably each R⁶ is independently selected from halogen, cyano, C₁-C₄haloalkyl and C₁-C₄haloalkoxy.

Embodiments according to the invention are provided as set out below.

Embodiment 1 provides a method of improving plant safety and/or reducing phytotoxicity and/or reducing plant necrosis whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), or a salt or N-oxide thereof, as defined above.

Embodiment 2 provides a method according to embodiment 1 wherein R¹ and R² are each independently C₁-C₄ alkyl.

Embodiment 3 provides a method according to embodiment 1 or 2 wherein R³ is C₁-C₃ alkyl.

Embodiment 4 provides a method according to any one of embodiments 1, 2 or 3 wherein R⁴ is C₁-C₄alkyl.

Embodiment 5 provides a method according to any one of embodiments 1, 2, 3 or 4 wherein R⁵ is phenyl (optionally substituted with one to three R⁶ groups), pyridyl (optionally substituted with one to three R⁶ groups) or thiazolyl (optionally substituted with one to three R⁶ groups) and each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₃-C₆cycloalkoxy, C₁-C₄alkylthio, C₁-C₄alkylsulfonyl, C₁-C₄haloalkylsulfonyl, C₁-C₄alkylcarbonyl, C₂-C₆alkenyl, C₂-C₆haloalkenyl, C₂-C₆alkynyl, phenyl, benzyl, phenoxy, pyridyl, pyridylmethyl and pyridyloxy.

Embodiment 6 provides a method according to any one of embodiments 1, 2, 3, 4 or 5 wherein R¹ and R² are each independently selected from methyl, ethyl and isopropyl.

Embodiment 7 provides a method according to any one of embodiments 1, 2, 3, 4, 5 or 6 wherein R³ is methyl, ethyl or isopropyl.

Embodiment 8 provides a method according to any one of embodiments 1, 2, 3, 4, 5, 6 or 7 wherein R⁴ is C₁-C₃alkyl.

Embodiment 9 provides a method according to any one of embodiments 1, 2, 3, 4, 5, 6, 7 or 8 wherein R⁵ is phenyl (optionally substituted with one or two R⁶ groups) or thiazolyl (optionally substituted with one or two R⁶ groups) and each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₁-C₄alkylthio, C₁-C₄alkylsulfonyl, C₁-C₄alkylcarbonyl, C₂-C₆alkynyl, phenyl, phenoxy and pyridyl.

Embodiment 10 provides a method according to any one of embodiments 1, 2, 3, 4, 5, 6, 7, 8 or 9 wherein R¹ and R² are each independently selected from methyl and ethyl.

Embodiment 11 provides a method according to any one of embodiments 1, 2, 3, 4, 5, 6, 7, 8, 9 or 10 wherein R³ is methyl or ethyl.

Embodiment 12 provides a method according to any one of embodiments 1, 2, 3, 4, 5, 6, 7, 8, 9, 10 or 11 wherein R⁴ is methyl or ethyl.

Embodiment 13 provides a method according to any one of embodiments 1, 2, 3, 4, 5, 6, 7, 8, 9, 10, 11 or 12 wherein R⁵ is phenyl (optionally substituted with one or two R⁶ groups) and each R⁶ is independently selected from halogen, cyano, C₁-C₄alkyl, C₁-C₄haloalkyl, C₁-C₄alkoxy, C₁-C₄haloalkoxy, C₂-C₆alkynyl and phenyl.

Embodiment 14 provides a method according to any one of embodiments 1, 2, 3, 4, 5, 6, 7, 8, 9, 10, 11, 12 or 13 wherein R¹ is methyl and R² is ethyl.

Embodiment 15 provides a method according to any one of embodiments 1, 2, 3, 4, 5, 6, 7, 8, 9, 10, 11, 12, 13 or 14 wherein R³ is methyl.

Embodiment 16 provides a method according to any one of embodiments 1, 2, 3, 4, 5, 6, 7, 8, 9, 10, 11, 12, 13, 14 or 15 wherein R⁴ is methyl.

Embodiment 17 provides a method according to any one of embodiments 1, 2, 3, 4, 5, 6, 7, 8, 9, 10, 11, 12, 13, 14, 15 or 16 wherein R⁵ is phenyl, which is optionally substituted by one or two substituents independently selected from trifluoromethyl and halogen (preferably fluoro or chloro).

Embodiment 18 provides a method according to any one of embodiments 1, 2, 3, 4, 5, 6, 7, 8, 9, 10, 11, 12, 13, 14, 15, 16 or 17 wherein both of R³ and R⁴ are methyl.

A preferred group of compounds of formula (I) are those of formula (I-1) which are compounds of formula (I) wherein R¹ and R² are each independently C₁-C₄ alkyl; R³ is C₁-C₃ alkyl; R⁴ is C₁-C₄alkyl; R⁵ is phenyl (optionally substituted with one to three R⁶ groups), pyridyl (optionally substituted with one to three R⁶ groups) or thiazolyl (optionally substituted with one to three R⁶ groups); and each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₃-C₆cycloalkoxy, C₁-C₄alkylthio, C₁-C₄alkylsulfonyl, C₁-C₄haloalkylsulfonyl, C₁-C₄alkylcarbonyl, C₂-C₆alkenyl, C₂-C₆haloalkenyl, C₂-C₆alkynyl, phenyl, benzyl, phenoxy, pyridyl, pyridylmethyl and pyridyloxy; or a salt or N-oxide thereof.

A further preferred group of compounds of formula (I) are those of formula (I-2) which are compounds of formula (I) wherein R¹ and R² are each independently selected from methyl, ethyl and isopropyl; R³ is methyl, ethyl or isopropyl; R⁴ is C₁-C₃alkyl; R⁵ is phenyl (optionally substituted with one or two R⁶ groups) or thiazolyl (optionally substituted with one or two R⁶ groups); and each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₁-C₄alkylthio, C₁-C₄alkylsulfonyl, C₁-C₄alkylcarbonyl, C₂-C₆alkynyl, phenyl, phenoxy and pyridyl; or a salt or N-oxide thereof.

A further preferred group of compounds of formula (I) are those of formula (I-3) which are compounds of formula (I) wherein R¹ and R² are each independently selected from methyl and ethyl; R³ is methyl or ethyl; R⁴ is methyl or ethyl; R⁵ is phenyl (optionally substituted with one or two R⁶ groups); and each R⁶ is independently selected from halogen, cyano, C₁-C₄alkyl, C₁-C₄haloalkyl, C₁-C₄alkoxy, C₁-C₄haloalkoxy, C₂-C₆alkynyl and phenyl; or a salt or N-oxide thereof.

A further preferred group of compounds of formula (I) are those of formula (I-4) which are compounds of formula (I) wherein R¹ is methyl and R² is ethyl; R³ is methyl; R⁴ is methyl; and R⁵ is phenyl, which is optionally substituted by one or two substituents independently selected from trifluoromethyl and halogen (preferably fluoro or chloro); or a salt or N-oxide thereof.

Certain compounds of formula (I) are novel and as such form a further aspect of the invention.

For example, there are provided novel compounds of formula (IH) which are compounds of formula (I) wherein R¹ and R² are each independently selected from C₁-C₄alkyl and C₃-C₈cycloalkyl; R³ is C₁-C₄ alkyl; R⁴ is C₁-C₄alkyl or C₁-C₄haloalkyl; R⁵ is aryl (optionally substituted with one to three R⁶ groups) or heteroaryl (optionally substituted with one to three R⁶ groups); and each R⁶ is independently selected from halogen, cyano, hydroxyl, amino, nitro, C₁-C₄alkyl, C₁-C₄haloalkyl, C₃-C₆cycloalkyl, C₃-C₆halocycloalkyl, C₁-C₄alkoxy, C₁-C₄haloalkoxy, C₃-C₆cycloalkoxy, C₁-C₄alkylthio, C₁-C₄haloalkylthio, C₃-C₆cycloalkylthio, C₁-C₄alkylsulfinyl, C₁-C₄haloalkylsulfinyl, C₁-C₄alkylsulfonyl, C₁-C₄haloalkylsulfonyl, C₁-C₄alkylcarbonyl, C₁-C₄alkoxycarbonyl, C₁-C₄alkylcarbonyloxy, C₂-C₆alkenyl, C₂-C₆haloalkenyl, C₂-C₆alkenyloxy, C₂-C₆haloalkenyloxy, C₂-C₆alkynyl, C₃-C₆cycloalkylC₂-C₆alkynyl, C₂-C₆alkynyloxy, aryl, aryl(C₁-C₄)alkyl, aryloxy, heteroaryl, heteroaryl(C₁-C₄)alkyl and heteroaryloxy; or a salt or an N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, 5-Chloro-3-(trifluoromethyl)phenyl, 4-Chloro-3-(isopropyl)phenyl, 4-Chloro-3-(tert-butyl)phenyl, or 5-cyclopropyl-1,3,4-thiadiazol-2-yl.

Preferred definitions of R¹, R², R³, R⁴, R⁵ and R⁶ are as defined above for compounds of formula (I), whilst taking account of any respective limitations in scope and the provisory clause.

A preferred group of novel compounds are those of formula (IHA) which are compounds of formula (IH) wherein R¹ and R² are each independently C₁-C₄ alkyl; R³ is C₁-C₃ alkyl; R⁴ is C₁-C₄alky; R⁵ is phenyl (optionally substituted with one to three R⁶ groups), pyridyl (optionally substituted with one to three R⁶ groups) or thiazolyl (optionally substituted with one to three R⁶ groups); and each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₃-C₆cycloalkoxy, C₁-C₄alkylthio, C₁-C₄alkylsulfonyl, C₁-C₄haloalkylsulfonyl, C₁-C₄alkylcarbonyl, C₂-C₆alkenyl, C₂-C₆haloalkenyl, C₂-C₆alkynyl, phenyl, benzyl, phenoxy, pyridyl, pyridylmethyl and pyridyloxy; or a salt or N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, 5-Chloro-3-(trifluoromethyl)phenyl, 4-Chloro-3-(isopropyl)phenyl, or 4-Chloro-3-(tert-butyl)phenyl.

Another preferred group of novel compounds are those of formula (IHB) which are compounds of formula (IH) wherein R¹ and R² are each independently selected from methyl, ethyl and isopropyl; R³ is methyl, ethyl or isopropyl; R⁴ is C₁-C₃alkyl; R⁵ is phenyl (optionally substituted with one or two R⁶ groups) or thiazolyl (optionally substituted with one or two R⁶ groups); and each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₁-C₄alkylthio, C₁-C₄alkylsulfonyl, C₁-C₄alkylcarbonyl, C₂-C₆alkynyl, phenyl, phenoxy and pyridyl; or a salt or N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, 5-Chloro-3-(trifluoromethyl)phenyl, 4-Chloro-3-(isopropyl)phenyl, or 4-Chloro-3-(tert-butyl)phenyl.

Another preferred group of novel compounds are those of formula (IHC) which are compounds of formula (IH) wherein R¹ and R² are each independently selected from methyl and ethyl; R³ is methyl or ethyl; R⁴ is methyl or ethyl; R⁵ is phenyl (optionally substituted with one or two R⁶ groups); and each R⁶ is independently selected from halogen, cyano, C₁-C₄alkyl, C₁-C₄haloalkyl, C₁-C₄alkoxy, C₁-C₄haloalkoxy, C₂-C₆alkynyl and phenyl; or a salt or N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, 5-Chloro-3-(trifluoromethyl)phenyl, 4-Chloro-3-(isopropyl)phenyl, or 4-Chloro-3-(tert-butyl)phenyl.

Another preferred group of novel compounds are those of formula (IHD) which are compounds of formula (IH) wherein R¹ is methyl and R² is ethyl; R³ is methyl; R⁴ is methyl; and R⁵ is phenyl, which is optionally substituted by one or two substituents independently selected from trifluoromethyl and halogen (preferably fluoro or chloro); or a salt or N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, or 5-Chloro-3-(trifluoromethyl)phenyl.

The following compounds are particularly preferred novel compounds of formula (I):

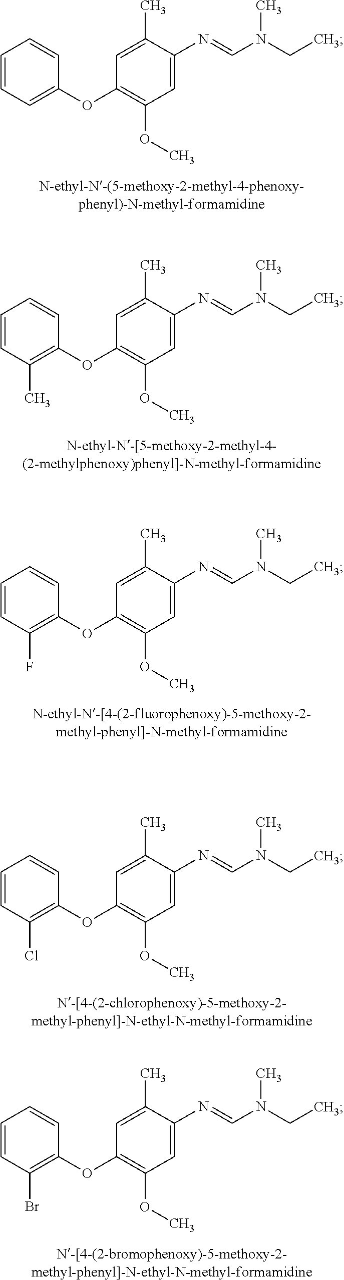 [SMILES: CCN(C)/C=N/c1cc(OC)c(Oc2ccccc2)cc1C ; CCN(C)/C=N/c1cc(OC)c(Oc2ccccc2C)cc1C ; CCN(C)/C=N/c1cc(OC)c(Oc2ccccc2F)cc1C ; CCN(C)/C=N/c1cc(OC)c(Oc2ccccc2Cl)cc1C ; CCN(C)/C=N/c1cc(OC)c(Oc2ccccc2Br)cc1C]

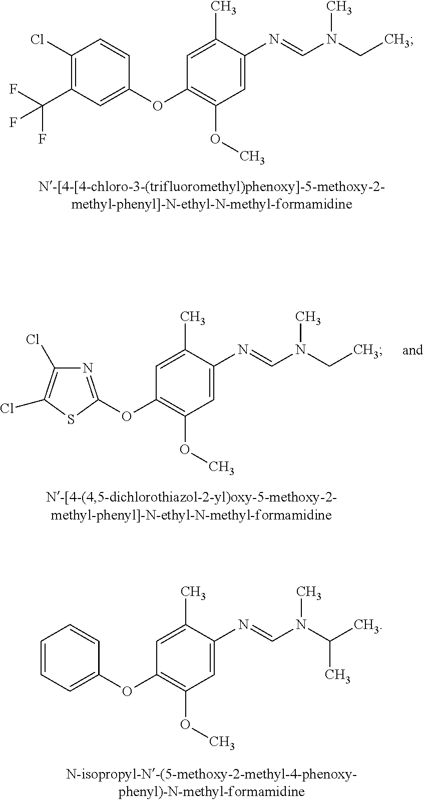 [SMILES: CCN(C)/C=N/c1cc(OC)c(Oc2ccc(Cl)c(C(F)(F)F)c2)cc1C ; CCN(C)/C=N/c1cc(OC)c(Oc2nc(Cl)c(Cl)s2)cc1C ; "and" ; COc1cc(/N=C/N(C)C(C)C)c(C)cc1Oc1ccccc1]

Other particularly preferred compounds are:

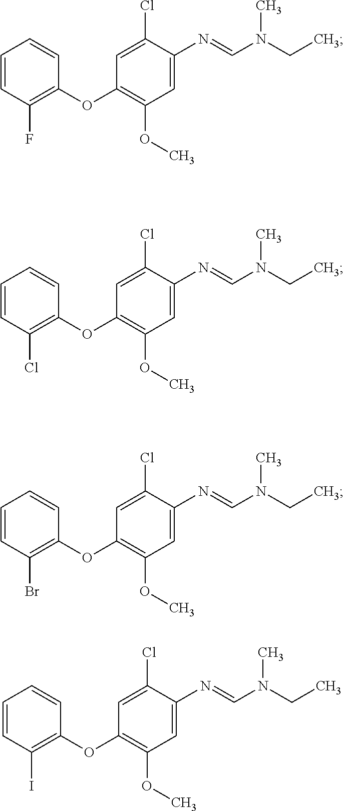 [SMILES: CCN(C)/C=N/c1cc(OC)c(Oc2ccccc2F)cc1Cl ; CCN(C)/C=N/c1cc(OC)c(Oc2ccccc2Cl)cc1Cl ; CCN(C)/C=N/c1cc(OC)c(Oc2ccccc2Br)cc1Cl ; CCN(C)/C=N/c1cc(OC)c(Oc2ccccc2I)cc1Cl]

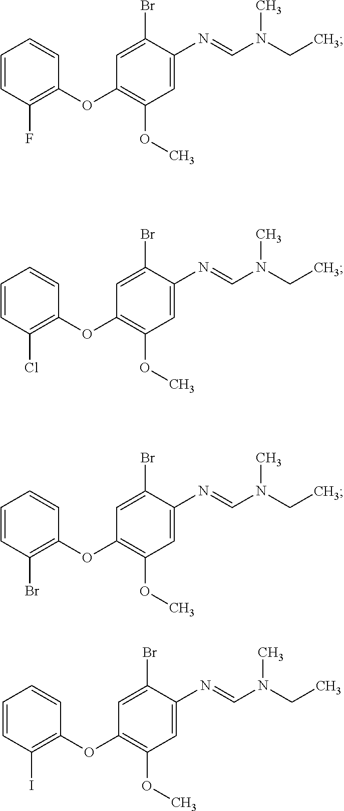 [SMILES: CCN(C)/C=N/c1cc(OC)c(Oc2ccccc2F)cc1Br ; CCN(C)/C=N/c1cc(OC)c(Oc2ccccc2Cl)cc1Br ; CCN(C)/C=N/c1cc(OC)c(Oc2ccccc2Br)cc1Br ; CCN(C)/C=N/c1cc(OC)c(Oc2ccccc2I)cc1Br]

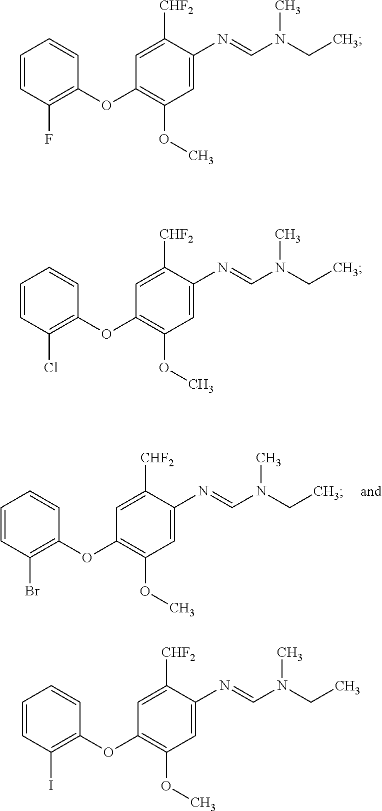

The structures of the following compounds are disclosed in WO08110278 (cf. compound numbers: 720, 721, 724, 725, 728, 729, 732, 733, 736 and 737) and are hereby excluded from the scope of the invention insofar as it relates to novel compounds of formula (I) (and insofar as said compounds disclosed in WO08110278 are enabled and made available):

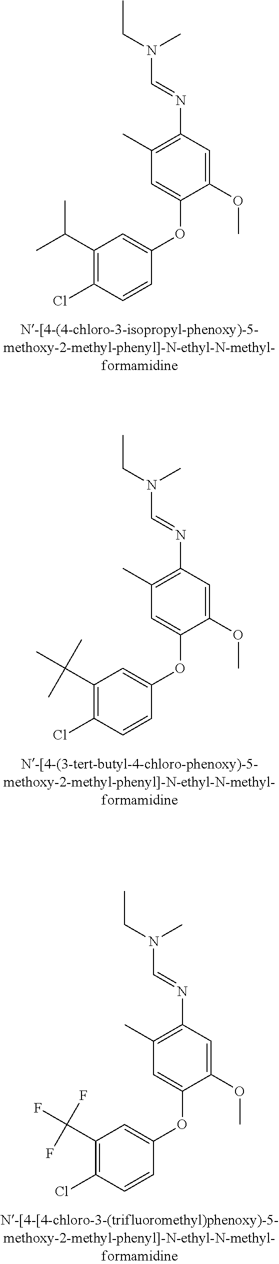

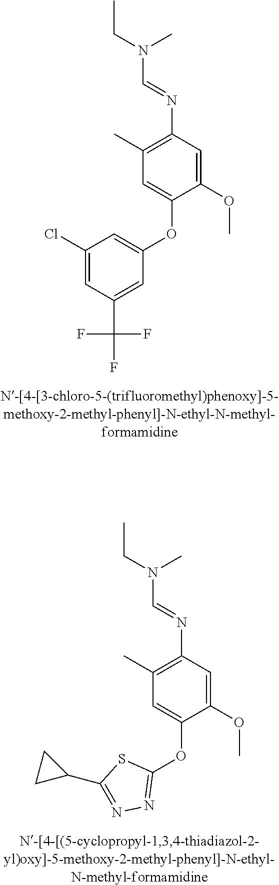 [SMILES: CCN(C)/C=N/c1cc(OC)c(Oc2cc(Cl)cc(C(F)(F)F)c2)cc1C ; CCN(C)/C=N/c1cc(OC)c(Oc2nnc(C3CC3)s2)cc1C]

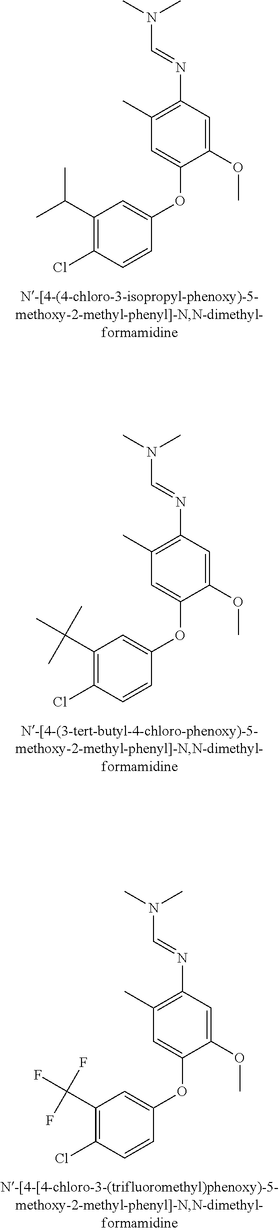

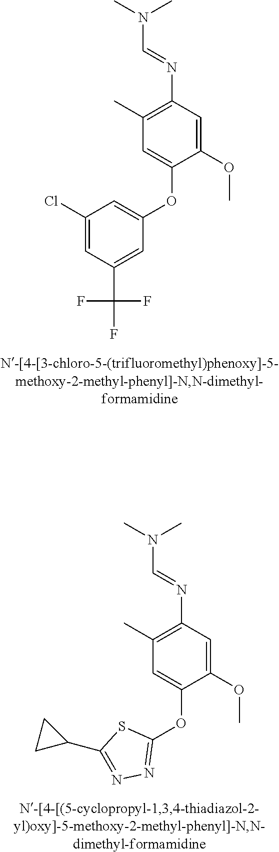 [SMILES: COc1cc(/N=C/N(C)C)c(C)cc1Oc1cc(Cl)cc(C(F)(F)F)c1 ; COc1cc(/N=C/N(C)C)c(C)cc1Oc1nnc(C2CC2)s1]

The above ten (10) compounds disclosed in WO08110278 are alleged to be herbicidal.

Compounds of formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile, improved physico-chemical properties, or increased biodegradability).

Specific examples of compounds of formula (I) are illustrated in the Tables 1 to 25 below.

Each of Tables 1 to 25, which follow the Table P below, make available 32 compounds of the formula (I-A)

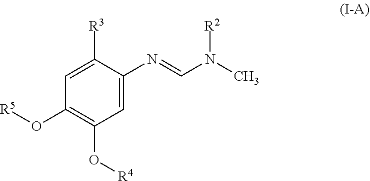

wherein R², R³ and R⁴ are as defined in Table P and R⁵ is as defined in Tables 1 to 25, respectively.

Thus Table 1 individualizes 32 compounds of formula (IA) wherein for each row of Table P, R⁴ is as defined in Table 1; similarly, Table 2 individualizes 32 compounds of formula (IA) wherein for each row of Table P, R³ is as defined in Table 2; and so on for Tables 3 to 25.

TABLE P

| Compound | | | |
|---|---|---|---|
| No | R² | R³ | R⁴ |
| P.001 | Me | Me | Me |
| P.002 | Me | Me | Et |
| P.003 | Me | Me | CHF₂ |
| P.004 | Me | Me | CF₃ |
| P.005 | Me | Et | Me |
| P.006 | Me | Et | Et |
| P.007 | Me | Et | CHF₂ |
| P.008 | Me | Et | CF₃ |
| P.009 | Et | Me | Me |
| P.010 | Et | Me | Et |
| P.011 | Et | Me | CHF₂ |
| P.012 | Et | Me | CF₃ |
| P.013 | Et | Et | Me |
| P.014 | Et | Et | Et |
| P.015 | Et | Et | CHF₂ |
| P.016 | Et | Et | CF₃ |
| P.017 | i-Pr | Me | Me |
| P.018 | i-Pr | Me | Et |
| P.019 | i-Pr | Me | CHF₂ |
| P.020 | i-Pr | Me | CF₃ |
| P.021 | i-Pr | Et | Me |
| P.022 | i-Pr | Et | Et |
| P.023 | i-Pr | Et | CHF₂ |
| P.024 | i-Pr | Et | CF₃ |
| P.025 | cyclopropyl | Me | Me |
| P.026 | cyclopropyl | Me | Et |
| P.027 | cyclopropyl | Me | CHF₂ |
| P.028 | cyclopropyl | Me | CF3 |
| P.029 | cyclopropyl | Et | Me |
| P.030 | cyclopropyl | Et | Et |
| P.031 | cyclopropyl | Et | CHF₂ |
| P.032 | cyclopropyl | Et | CF₃ |

Table 1: This table discloses 32 compounds 1.001 to 1.032 of the formula IA wherein R⁵ is

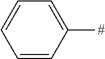

wherein the hash mark indicates the point of attachment of R⁵ to the rest of the molecule, and in which the variables R², R³ and R⁴ have the specific meaning given in the corresponding line of Table P. For example, compound 1.001 has the following structure:

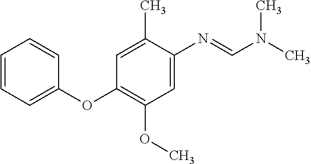

Table 2: This table discloses 32 compounds 2.001 to 2.032 of the formula IA wherein R⁵ is

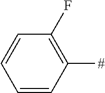

wherein the hash mark indicates the point of attachment of R⁵ to the rest of the molecule, and in which the variables R², R³ and R⁴ have the specific meaning given in the corresponding line of Table P.

Table 3: This table discloses 32 compounds 3.001 to 3.032 of the formula IA wherein R⁵ is

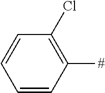

wherein the hash mark indicates the point of attachment of R⁵ to the rest of the molecule, and in which the variables R², R³ and R⁴ have the specific meaning given in the corresponding line of Table P.

Table 4: This table discloses 32 compounds 4.001 to 4.032 of the formula IA wherein R⁵ is

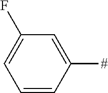

wherein the hash mark indicates the point of attachment of R⁵ to the rest of the molecule, and in which the variables R², R³ and R⁴ have the specific meaning given in the corresponding line of Table P.

Table 5: This table discloses 32 compounds 5.001 to 5.032 of the formula IA wherein R⁵ is

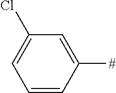

wherein the hash mark indicates the point of attachment of R⁵ to the rest of the molecule, and in which the variables R², R³ and R⁴ have the specific meaning given in the corresponding line of Table P.

Table 6: This table discloses 32 compounds 6.001 to 6.032 of the formula IA wherein R⁵ is

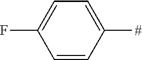

wherein the hash mark indicates the point of attachment of R⁵ to the rest of the molecule, and in which the variables R², R³ and R⁴ have the specific meaning given in the corresponding line of Table P.

Table 7: This table discloses 32 compounds 7.001 to 7.032 of the formula IA wherein R⁵ is

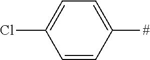

wherein the hash mark indicates the point of attachment of R⁵ to the rest of the molecule, and in which the variables R², R³ and R⁴ have the specific meaning given in the corresponding line of Table P.

Table 8: This table discloses 32 compounds 8.001 to 8.032 of the formula IA wherein R⁵ is

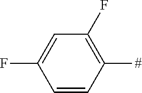

wherein the hash mark indicates the point of attachment of R⁵ to the rest of the molecule, and in which the variables R², R³ and R⁴ have the specific meaning given in the corresponding line of Table P.

Table 9: This table discloses 32 compounds 9.001 to 9.032 of the formula IA wherein R⁵ is

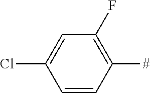

wherein the hash mark indicates the point of attachment of R⁵ to the rest of the molecule, and in which the variables R², R³ and R⁴ have the specific meaning given in the corresponding line of Table P.

Table 10: This table discloses 32 compounds 10.001 to 10.032 of the formula IA wherein R⁵ is

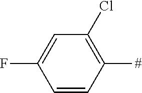

wherein the hash mark indicates the point of attachment of R⁵ to the rest of the molecule, and in which the variables R², R³ and R⁴ have the specific meaning given in the corresponding line of Table P.

Table 11: This table discloses 32 compounds 11.001 to 11.032 of the formula IA wherein R⁵ is

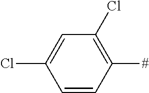

wherein the hash mark indicates the point of attachment of R⁵ to the rest of the molecule, and in which the variables R², R³ and R⁴ have the specific meaning given in the corresponding line of Table P.

Table 12: This table discloses 32 compounds 12.001 to 12.032 of the formula IA wherein R⁵ is

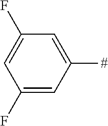

wherein the hash mark indicates the point of attachment of R⁵ to the rest of the molecule, and in which the variables R², R³ and R⁴ have the specific meaning given in the corresponding line of Table P.

Table 13: This table discloses 32 compounds 13.001 to 13.032 of the formula IA wherein R⁵ is

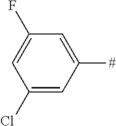

wherein the hash mark indicates the point of attachment of R⁵ to the rest of the molecule, and in which the variables R², R³ and R⁴ have the specific meaning given in the corresponding line of Table P.

Table 14: This table discloses 32 compounds 14.001 to 14.032 of the formula IA wherein R⁵ is

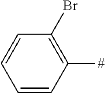

wherein the hash mark indicates the point of attachment of R⁵ to the rest of the molecule, and in which the variables R², R³ and R⁴ have the specific meaning given in the corresponding line of Table P.

Table 15: This table discloses 32 compounds 15.001 to 15.032 of the formula IA wherein R⁵ is

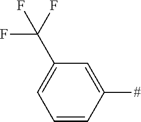

wherein the hash mark indicates the point of attachment of R⁵ to the rest of the molecule, and in which the variables R², R³ and R⁴ have the specific meaning given in the corresponding line of Table P.

Table 16: This table discloses 32 compounds 16.001 to 16.032 of the formula IA wherein R⁵ is

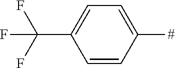

wherein the hash mark indicates the point of attachment of R⁵ to the rest of the molecule, and in which the variables R², R³ and R⁴ have the specific meaning given in the corresponding line of Table P.

Table 17: This table discloses 32 compounds 17.001 to 17.032 of the formula IA wherein R⁵ is

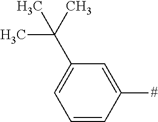

wherein the hash mark indicates the point of attachment of R⁵ to the rest of the molecule, and in which the variables R², R³ and R⁴ have the specific meaning given in the corresponding line of Table P.

Table 18: This table discloses 32 compounds 18.001 to 18.032 of the formula IA wherein R⁵ is

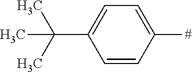

wherein the hash mark indicates the point of attachment of R⁵ to the rest of the molecule, and in which the variables R², R³ and R⁴ have the specific meaning given in the corresponding line of Table P.

Table 19: This table discloses 32 compounds 19.001 to 19.032 of the formula IA wherein R⁵ is

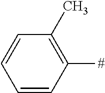

wherein the hash mark indicates the point of attachment of R⁵ to the rest of the molecule, and in which the variables R², R³ and R⁴ have the specific meaning given in the corresponding line of Table P.

Table 20: This table discloses 32 compounds 20.001 to 20.032 of the formula IA wherein R⁵ is

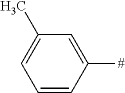

wherein the hash mark indicates the point of attachment of R⁵ to the rest of the molecule, and in which the variables R², R³ and R⁴ have the specific meaning given in the corresponding line of Table P.

Table 21: This table discloses 64 compounds 21.001 to 22.032 of the formula IA wherein R⁵ is

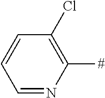

wherein the hash mark indicates the point of attachment of R⁵ to the rest of the molecule, and in which the variables R², R³ and R⁴ have the specific meaning given in the corresponding line of Table P.

Table 22: This table discloses 64 compounds 22.001 to 22.032 of the formula IA wherein R⁵ is

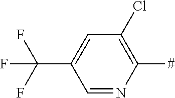

wherein the hash mark indicates the point of attachment of R⁵ to the rest of the molecule, and in which the variables R², R³ and R⁴ have the specific meaning given in the corresponding line of Table P.

Table 23: This table discloses 32 compounds 24.001 to 24.032 of the formula IA wherein R⁵ is

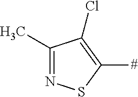

wherein the hash mark indicates the point of attachment of R⁵ to the rest of the molecule, and in which the variables R², R³ and R⁴ have the specific meaning given in the corresponding line of Table P.

Table 24: This table discloses 32 compounds 24.001 to 24.032 of the formula IA wherein R⁵ is

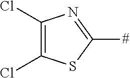

wherein the hash mark indicates the point of attachment of R⁵ to the rest of the molecule, and in which the variables R², R³ and R⁴ have the specific meaning given in the corresponding line of Table P.

Table 25: This table discloses 32 compounds 25.001 to 25.032 of the formula IA wherein R⁵ is

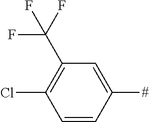

wherein the hash mark indicates the point of attachment of R⁵ to the rest of the molecule, and in which the variables R², R³ and R⁴ have the specific meaning given in the corresponding line of Table P.

Compounds of formula (I) can be made as shown in the following schemes, in which, unless otherwise stated, the definition of each variable is as defined for a compound of formula (I).

The compounds of formula (I), wherein R¹, R², R³, R⁴ and R⁵ are as defined above for compounds of formula (I), can be obtained by transformation of a compound of formula (II), wherein R³, R⁴ and R⁵ are as defined for compounds of formula (I), by several known methods among which the most widely uses are the following:
a) Treatment with a compound of formula (III-a), wherein R¹ and R² are as defined for compounds of formula (I) and R⁷ is C₁-C₄alkyl, in an organic solvent such as toluene or methanol at temperatures between 0° C. and 100° C.
b) Treatment with an orthoester of formula (III-b), wherein R⁷ is C₁-C₄alkyl, followed by treatment with an amine of formula (III-c), wherein R¹ and R² are as defined for compounds of formula (I), in an organic solvent such as methanol at temperatures between 20° C. and 100° C.
c) Treatment with a formamide of formula (III-d), wherein R¹ and R² are as defined for compounds of formula (I), and an activating agent such as POCl₃ in an inert solvent such as dichloromethane at temperatures between −20° C. and 40° C.
   This is shown in Scheme 1 below.

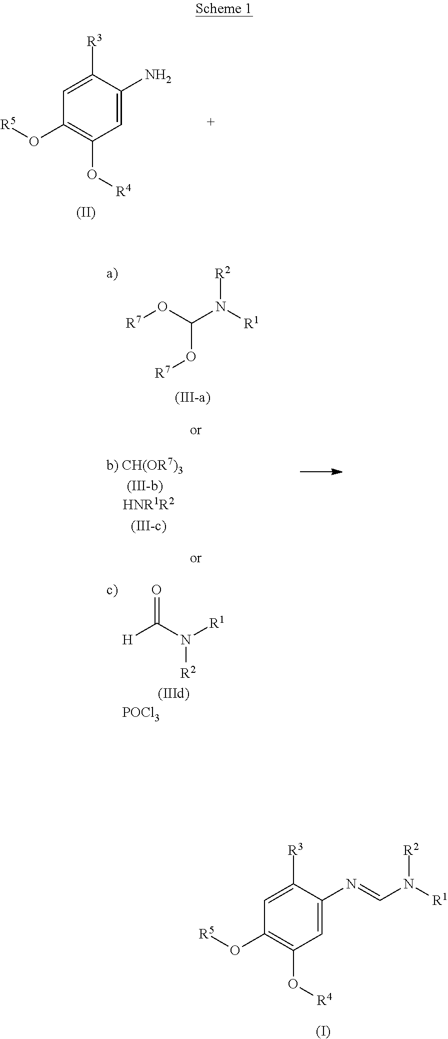

Compounds of formula (II), wherein R³, R⁴ and R⁵ are as defined for compounds of formula (I), can be obtained through reduction of a nitro compound of formula (III), wherein R³, R⁴ and R⁵ are as defined for compounds of formula (I), by numerous methods among which most preferably applied are the treatment with a) a metal, preferably Zn or Fe, in the presence of a proton source, preferably acetic acid, NH₄Cl or HCl, in a solvent such as ethanol or acetic acid at temperature between 20° C. and 120° C. b) a transition metal based catalyst such as Pd, Co, Pt or Ni in the presence of hydrogen gas or an equivalent thereof such as ammonium formate or sodium borohydride, in an inert solvent such as methanol at temperatures between 20° C. and 100° C. This is shown in Scheme 2 below.

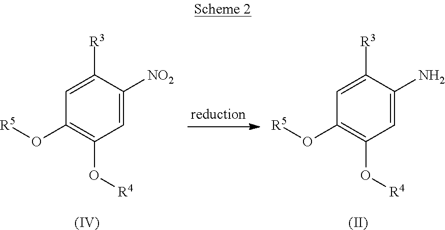

Compounds of formula (IV), wherein R³, R⁴ and R⁵ are as defined for compounds of formula (I), can be obtained by transformation of a compound of formula (V), wherein R³ and R⁴ are as defined for compounds of formula (I) and Hal is halogen, with a compound of formula (VI), wherein R⁵ is as defined for compounds of formula (I). This is shown in Scheme 3 below.

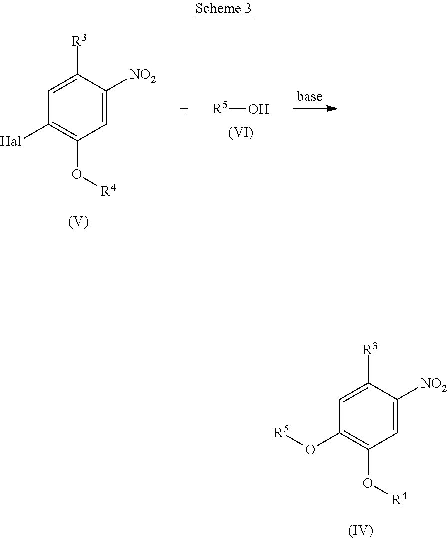

Alternatively, compounds of formula (IV), wherein R³, R⁴ and R⁵ are as defined for compounds of formula (I), can be obtained by transformation of a compound of formula (VII), wherein R³ and R⁴ are as defined for compounds of formula (I), with a compound of formula (VIII), wherein R⁵ is as defined for compounds of formula (I) and X is halogen, such as fluoro, chloro or bromo, or a sulfonate, such as mesylate. This is shown in Scheme 4 below.

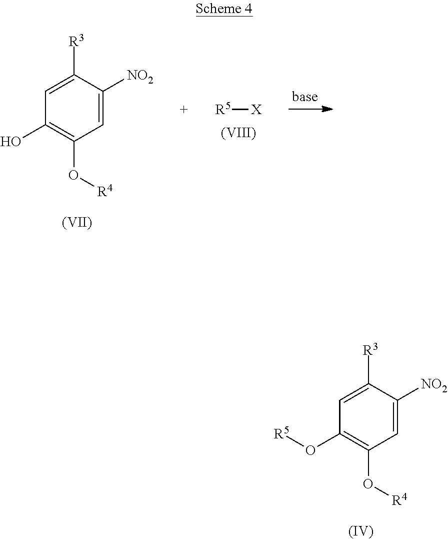

Compounds of formula (V), wherein R³ and R⁴ are as defined for compounds of formula (I) and Hal is halogen, can be obtained by alkylation of a compound of formula (IX), wherein R³ is as defined for compounds of formula (I) and Hal is halogen, with a compound of formula (X), wherein R⁴ is as defined for compounds of formula (I) and X is halogen, such as chloro, bromo or iodo, or a sulfonate, such as mesylate. This is shown in Scheme 5 below.

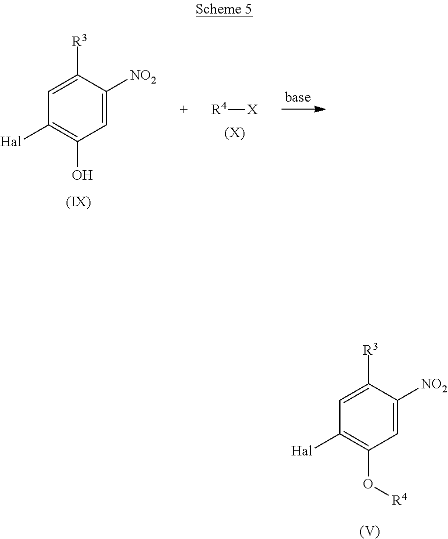

Compounds of formula (IX), wherein R³ is as defined for compounds of formula (I) and Hal is halogen, can be obtained by nitration of a compound of formula (XI), wherein R³ is as defined for compounds of formula (I) and Hal is halogen. This is described in J. Med. Chem. 2015, 8413-8426 and shown in Scheme 6 below.

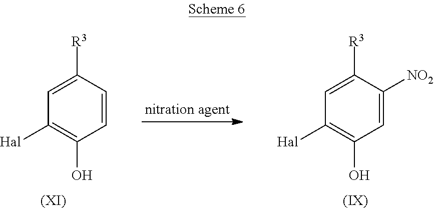

Compounds of formula (VII), wherein R³ and R⁴ are as defined for compounds of formula (I), can be obtained by transformation of a compound of formula (XII), wherein R³ and R⁴ are as defined for compounds of formula (I) and R⁷ is C₁-C₄alkyl, with a nucleophile such as LiCl or sodium ethane thiolate, in an inert solvent such as DMF. This is shown in Scheme 7 below.

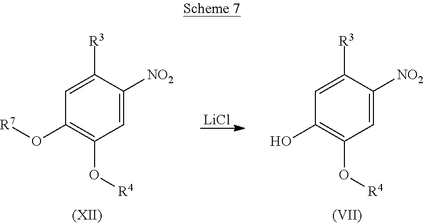

Compounds of formula (XII), wherein R³ and R⁴ are as defined for compounds of formula (I) and R⁷ is C₁-C₄alkyl, can be obtained by transformation of a compound of formula (XIII), wherein R³ and R⁴ are as defined for compounds of formula (I) and R⁷ is C₁-C₄alkyl. This is shown in Scheme 8 below.

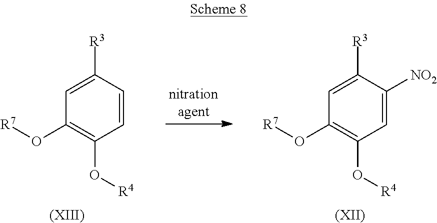

Alternatively, compounds of formula (I), wherein R¹, R², R³, R⁴ and R⁵ are as defined for compounds of formula (I), can be obtained by transformation of a compound of formula (XIV), wherein R¹, R², R³ and R⁴ are as defined for compounds of formula (I) and Hal is halogen such as chloro or bromo, with a compound of formula (VI), wherein R⁵ is as defined for compounds of formula (I), in the presence of a suitably supported, Pd or Cu based transition metal based catalyst and a base. This is shown in Scheme 9 below.

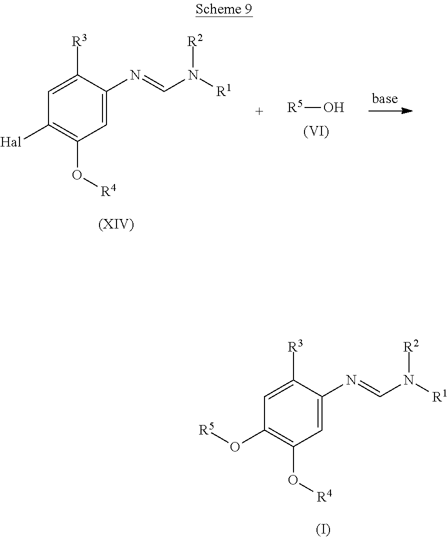

Compounds of formula (XIV), wherein R¹, R², R³ and R⁴ are as defined for compounds of formula (I) and Hal is halogen, can be obtained by transformation of a compound of formula (XV), wherein R³ and R⁴ are as defined for compounds of formula (I) and Hal is halogen, by several known methods among which the most widely uses are the following:
a) Treatment with a compound of formula (III-a), wherein R¹ and R² are as defined for compounds of formula (I) and R⁷ is C₁-C₄alkyl, in an inert solvent such as toluene at temperatures between 0° C. and 100° C.
b) Treatment with an orthoester of formula (III-b), wherein R⁷ is C₁-C₄alkyl, followed by treatment with an amine of formula (III-c), wherein R¹ and R² are as defined for compounds of formula (I), in an organic solvent such as methanol at temperatures between 20° C. and 100° C.
c) Treatment with a formamide of formula (III-d), wherein R¹ and R² are as defined for compounds of formula (I), and an activating agent such as POCl₃in an inert solvent such as dichloromethane at temperatures between −20° C. and 40° C.
   This is shown in Scheme 10 below.

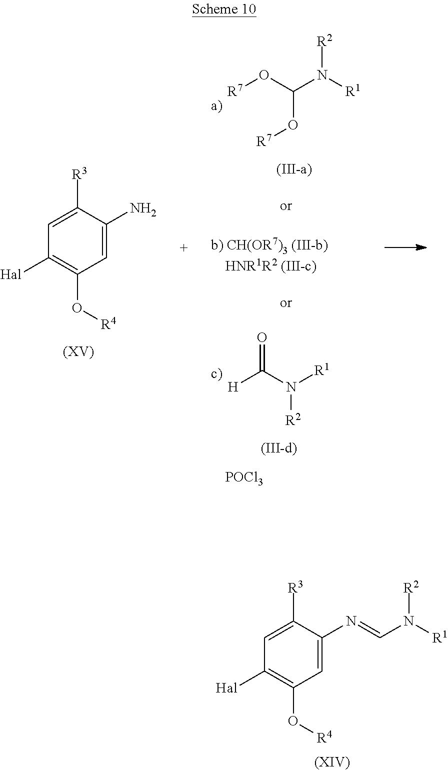

Compounds of formula (XV), wherein R³ and R⁴ are as defined for compounds of formula (I) and Hal is halogen, can be obtained by transformation of a compound of formula (XVI), wherein R³ and R⁴ are as defined for compounds of formula (I). This is shown in Scheme 11 below.

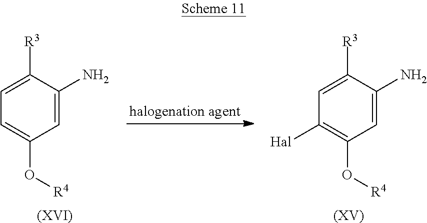

Certain intermediates described in the above schemes are novel and as such form a further aspect of the invention.

The compounds of formula (I) can be used in the agricultural sector and related fields of use e.g. as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and may be used for protecting numerous cultivated plants. The compounds of formula (I) can be used to inhibit or destroy the pests that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula (I) as fungicide. The term “fungicide” as used herein means a compound that controls, modifies, or prevents the growth of fungi. The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It is also possible to use compounds of formula (I) as dressing agents for the treatment of plant propagation material, e.g., seed, such as fruits, tubers or grains, or plant cuttings (for example rice), for the protection against fungal infections as well as against phytopathogenic fungi occurring in the soil. The propagation material can be treated with a composition comprising a compound of formula (I) before planting: seed, for example, can be dressed before being sown. The compounds of formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore the compounds of formula (I) can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

Compounds of formula (I) and fungicidal compositions containing them may be used to control plant diseases caused by a broad spectrum of fungal plant pathogens. They are effective in controlling a broad spectrum of plant diseases, such as foliar pathogens of ornamental, turf, vegetable, field, cereal, and fruit crops.

These fungi and fungal vectors of disease, as well as phytopathogenic bacteria and viruses, which may be controlled are for example:

Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea,

Coccidioides immitis, Cochliobolus spp, Colletotrichum spp. including C. musae,

Cryptococcus neoformans, Diaporthe spp, Didymella spp, Drechslera spp, Elsinoe spp,

Epidermophyton spp, Erwinia amylovora, Erysiphe spp. including E. cichoracearum,

Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi,

Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp, Stereum hirsutum, Thanatephorus cucumeris, Thielaviopsis basicola, Tilletia spp, Trichoderma spp. including T. harzianum, T. pseudokoningii, T. viride,

Trichophyton spp, Typhula spp, Uncinula necator, Urocystis spp, Ustilago spp, Venturia spp. including V. inaequalis, Verticillium spp, and Xanthomonas spp.

In particular, compounds of formula (I) and fungicidal compositions containing them may be used to control plant diseases caused by a broad spectrum of fungal plant pathogens in the Basidiomycete, Ascomycete, Oomycete and/or Deuteromycete, Blasocladiomycete, Chrytidiomycete, Glomeromycete and/or Mucoromycete classes.

These pathogens may include:

Oomycetes, including Phytophthora diseases such as those caused by Phytophthora capsici, Phytophthora infestans, Phytophthora sojae, Phytophthora fragariae, Phytophthora nicotianae, Phytophthora cinnamomi, Phytophthora citricola, Phytophthora citrophthora and Phytophthora erythroseptica; Pythium diseases such as those caused by Pythium aphanidermaturn, Pythium arrhenomanes, Pythium graminicola, Pythium irregulare and Pythium ultimum; diseases caused by Peronosporales such as Peronospora destructor, Peronospora parasitica, Plasmopara viticola, Plasmopara halstedii, Pseudoperonospora cubensis, Albugo candida, Sclerophthora macrospora and Bremia lactucae; and others such as Aphanomyces cochlioides, Labyrinthula zosterae, Peronosclerospora sorghi and Sclerospora graminicola.

Ascomycetes, including blotch, spot, blast or blight diseases and/or rots for example those caused by Pleosporales such as Stemphylium solani, Stagonospora tainanensis, Spilocaea oleaginea, Setosphaeria turcica, Pyrenochaeta lycoperisici, Pleospora herbarum, Phoma destructiva, Phaeosphaeria herpotrichoides, Phaeocryptocus gaeumannii, Ophiosphaerella graminicola, Ophiobolus graminis, Leptosphaeria maculans, Hendersonia creberrima, Helminthosporium triticirepentis, Setosphaeria turcica, Drechslera glycines, Didymella bryoniae, Cycloconium oleagineum, Corynespora cassiicola, Cochliobolus sativus, Bipolaris cactivora, Venturia inaequalis, Pyrenophora teres, Pyrenophora tritici-repentis, Alternaria alternata, Alternaria brassicicola, Alternaria solani and Alternaria tomatophila, Capnodiales such as Septoria tritici, Septoria nodorum, Septoria glycines, Cercospora arachidicola, Cercospora sojina, Cercospora zeae-maydis, Cercosporella capsellae and Cercosporella herpotrichoides, Cladosporium carpophilum, Cladosporium effusum, Passalora fulva, Cladosporium oxysporum, Dothistroma septosporum, Isariopsis clavispora, Mycosphaerella fijiensis, Mycosphaerella graminicola, Mycovellosiella koepkeii, Phaeoisariopsis bataticola, Pseudocercospora vitis, Pseudocercosporella herpotrichoides, Ramularia beticola, Ramularia collo-cygni, Magnaporthales such as Gaeumannomyces graminis, Magnaporthe grisea, Pyricularia oryzae, Diaporthales such as Anisogramma anomala, Apiognomonia errabunda, Cytospora platani, Diaporthe phaseolorum, Discula destructiva, Gnomonia fructicola, Greeneria uvicola, Melanconium juglandinum, Phomopsis viticola, Sirococcus clavigignenti-juglandacearum, Tubakia dryina, Dicarpella spp., Valsa ceratosperma, and others such as Actinothyrium graminis, Ascochyta pisi, Aspergillus flavus, Aspergillus fumigatus, Aspergillus nidulans, Asperisporium caricae, Blumeriella jaapii, Candida spp., Capnodium ramosum, Cephaloascus spp., Cephalosporium gramineum, Ceratocystis paradoxa, Chaetomium spp., Hymenoscyphus pseudoalbidus, Coccidioides spp., Cylindrosporium padi, Diplocarpon malae, Drepanopeziza campestris, Elsinoe ampelina, Epicoccum nigrum, Epidermophyton spp., Eutypa lata, Geotrichum candidum, Gibellina cerealis, Gloeocercospora sorghi, Gloeodes pomigena, Gloeosporium perennans; Gloeotinia temulenta, Griphospaeria corticola, Kabatiella lini, Leptographium microsporum, Leptosphaerulinia crassiasca, Lophodermium seditiosum, Marssonina graminicola, Microdochium nivale, Monilinia fructicola, Monographella albescens, Monosporascus cannonballus, Naemacyclus spp., Ophiostoma novo-ulmi, Paracoccidioides brasiliensis, Penicillium expansum, Pestalotia rhododendri, Petrieffidium spp., Pezicula spp., Phialophora gregata, Phyllachora pomigena, Phymatotrichum omnivora, Physalospora abdita, Plectosporium tabacinum, Polyscytalum pustulans, Pseudopeziza medicaginis, Pyrenopeziza brassicae, Ramulispora sorghi, Rhabdocline pseudotsugae, Rhynchosporium secalis, Sacrocladium oryzae, Scedosporium spp., Schizothyrium pomi, Sclerotinia sclerotiorum, Sclerotinia minor, Sclerotium spp., Typhula ishikariensis, Seimatosporium mariae, Lepteutypa cupressi, Septocyta ruborum, Sphaceloma perseae, Sporonema phacidioides, Stigmina palmivora, Tapesia yallundae, Taphrina bullata, Thielviopsis basicola, Trichoseptoria fructigena, Zygophiala jamaicensis; powdery mildew diseases for example those caused by Erysiphales such as Blumeria graminis, Erysiphe polygoni, Uncinula necator, Sphaerotheca fuligena, Podosphaera leucotricha, Podospaera macularis Golovinomyces cichoracearum, Leveillula taurica, Microsphaera diffusa, Oidiopsis gossypii, Phyllactinia guttata and Oidium arachidis; molds for example those caused by Botryosphaeriales such as Dothiorella aromatica, Diplodia seriata, Guignardia bidwellii, Botrytis cinerea, Botryotinia allii, Botryotinia fabae, Fusicoccum amygdali, Lasiodiplodia theobromae, Macrophoma theicola, Macrophomina phaseolina, Phyllosticta cucurbitacearum; anthracnoses for example those caused by Glommerelales such as Colletotrichum gloeosporioides, Colletotrichum lagenarium, Colletotrichum gossypii, Glomerella cingulata, and Colletotrichum graminicola; and wilts or blights for example those caused by Hypocreales such as Acremonium strictum, Claviceps purpurea, Fusarium culmorum, Fusarium graminearum, Fusarium virguliforme, Fusarium oxysporum, Fusarium subglutinans, Fusarium oxysporum f.sp. cubense, Gerlachia nivale, Gibberella fujikuroi, Gibberella zeae, Gliocladium spp., Myrothecium verrucaria, Nectria ramulariae, Trichoderma viride, Trichothecium roseum, and Verticillium theobromae.

Basidiomycetes, including smuts for example those caused by Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f.sp. Hordei, Puccinia striiformis f.sp. Secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries.

Blastocladiomycetes, such as Physoderma maydis.

Mucoromycetes, such as Choanephora cucurbitarum; Mucor spp.; Rhizopus arrhizus,

As well as diseases caused by other species and genera closely related to those listed above.

In addition to their fungicidal activity, the compounds and compositions comprising them may also have activity against bacteria such as Erwinia amylovora, Erwinia caratovora, Xanthomonas campestris, Pseudomonas syringae, Streptomyces scabies and other related species as well as certain protozoa.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The useful plants and/or target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The term “useful plants” and/or “target crops” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” is to be understood as including those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include δ-endotoxins, vegetative insecticidal proteins (Vip), insecticidal proteins of bacteria colonising nematodes, and toxins produced by scorpions, arachnids, wasps and fungi. An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

The term “useful plants” and/or “target crops” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Toxins that can be expressed by transgenic plants include, for example, insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO03/018810).

More examples of such toxins or transgenic plants capable of synthesising such toxins are disclosed, for example, in EP-A-0 374 753, WO93/07278, WO5/34656, EP-A-0 427 529, EP-A-451 878 and WO03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Pesticidal agents referred to herein using their common name are known, for example, from “The Pesticide Manual”, 15th Ed., British Crop Protection Council 2009.

The compounds of formula (I) may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

The invention also provides for the use of provides a composition comprising a compound of formula (I) according to the present invention together with one or more pesticides, plant nutrients or plant fertilizers. The combination may also encompass specific plant traits incorporated into the plant using any means, for example conventional breeding or genetic modification.

Suitable examples of plant nutrients or plant fertilizers are calcium sulfate (CaSO₄), calcium nitrate (Ca(NO₃)₂.4H₂O), calcium carbonate (CaCO₃), potassium nitrate (KNO₃), magnesium sulfate (MgSO₄), potassium hydrogen phosphate (KH₂PO₄), manganese sulfate (MnSO₄), copper sulfate (CuSO₄), zinc sulfate (ZnSO₄), nickel chloride (NiCl₂), cobalt sulfate CoSO₄), potassium hydroxide (KOH), sodium chloride (NaCl), boric acid (H₃BO₃) and metal salts thereof (Na₂MoO₄). The nutrients may be present in an amount of 5% to 50% by weight, preferably of 10% to 25% by weight or of 15% to 20% by weight each. Preferred additional nutrients are urea ((NH₂)₂CO), melamine (C₃H₆N₆), potassium oxide (K₂O), and inorganic nitrates. The most preferred additional plant nutrient is potassium oxide. Where the preferred additional nutrient is urea, it is present in an amount of generally 1% to 20% by weight, preferably 2% to 10% by weight or of 3% to 7% by weight.

Suitable examples of pesticides are acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, zinc fungicides, Benzoylureas, carbamates, chloronicotinyls, diacylhydrazines, diamides, fiproles, macrolides, nitroimines, nitromethylenes, organochlorines, organophosphates, organosilicons, organotins, phenylpyrazoles, phosphoric esters, pyrethroids, spinosyns, tetramic acid derivatives, tetronic acid derivatives, Antibiotic nematicides, avermectin nematicides, botanical nematicides, carbamate nematicides, oxime carbamate nematicides, organophosphorus nematicides, nematophagous fungi or bacteria, amide herbicides, anilide herbicides, arsenical herbicides, arylalanine herbicides, aryloxyphenoxypropionic herbicides, benzofuranyl herbicides, benzoic acid herbicides, benzothiazole herbicides, benzoylcyclohexanedione herbicides, carbamate herbicides, carbanilate herbicides, chloroacetanilide herbicides, chlorotriazine herbicides, cyclohexene oxmie herbicides, cyclopropylisoxazole herbicides, dicarboximide herbicides, dinitroaniline herbicides, dinitrophenol herbicides, diphenyl ether herbicides, dithiocarbamate herbicides, fluoroalkyltriazine herbicides, halogenated aliphatic herbicides, imidazolinone herbicides, inorganic herbicides, methoxytriazine herbicides, methylthiotriazine herbicides, nitrile herbicides, nitrophenyl ether herbicides, organophosphorous herbicides, oxadiazolone herbicides, oxazole herbicides, phenoxy herbicides, phenoxyacetic herbicides, phenoxybutyric herbicides, phenoxypropionic herbicides, phenylenediamine herbicides, phenylurea herbicides, phthalic acid herbicides, picolinic acid herbicides, pyrazole herbicides, pyridazine herbicides, pyridazinone herbicides, pyridine herbicides, pyrimidinediamine herbicides, pyrimidinyloxybenzylamine herbicides, pyrimidinylsulfonylurea herbicides, quaternary ammonium herbicides, quinolinecarboxylic acid herbicides, sulfonamide herbicides, sulfonanilide herbicides, sulfonylurea herbicides, thiadiazolylurea herbicides, thioamide herbicides, thiocarbamate herbicides, thiocarbonate herbicides, thiourea herbicides, triazine herbicides, triazinone herbicides, triazinylsulfonylurea herbicides, triazole herbicides, triazolone herbicides, triazolopyrimidine herbicides, uracil herbicides, urea herbicides, microbials, plant extracts, pheromones, macrobials and other biologicals.

A further aspect of invention is related to a method of controlling or preventing an infestation of plants, e.g. useful plants such as crop plants, propagation material thereof, e.g. seeds, harvested crops, e.g. harvested food crops, or of non-living materials by insects or by phytopathogenic or spoilage microorganisms or organisms potentially harmful to man, especially fungal organisms, which comprises the application of a compound of formula (I) or of a preferred individual compound as above-defined as active ingredient to the plants, to parts of the plants or to the locus thereof, to the propagation material thereof, or to any part of the non-living materials.

Controlling or preventing means reducing infestation by insects or by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I), or an agrochemical composition which contains at least one of said compounds, is foliar application. The frequency of application and the rate of application will depend on the risk of infestation by the corresponding pathogen or insect. However, the compounds of formula (I) can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula (I) may also be applied to seeds (coating) by impregnating the seeds or tubers either with a liquid formulation of the fungicide or coating them with a solid formulation.

A formulation, e.g. a composition containing the compound of formula (I), and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is preferably 1 g to 2000 g of active ingredient per hectare, more preferably 10 to 1000 g/ha, most preferably 10 to 600 g/ha. When used as seed drenching agent, convenient dosages are from 10 mg to 1 g of active substance per kg of seeds.

When the compositions are used for treating seed, rates of 0.001 to 50 g of a compound of formula (I) per kg of seed, preferably from 0.01 to 10 g per kg of seed are generally sufficient.

Suitably, a composition comprising a compound of formula (I) according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) together with component (B) and (C), and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

### EXAMPLES

The Examples which follow serve to illustrate the invention. Certain compounds of formula (I) can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

Throughout this description, temperatures are given in degrees Celsius and “m.p.” means melting point. LC/MS means Liquid Chromatography Mass Spectroscopy and the description of the apparatus and the methods are:

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

| Emulsifiable concentrate | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

| Extruder granules | |
|---|---|
| Active ingredient [compound of formula (I)] | 15% |
| sodium lignosulfonate | 2% |
| carboxymethylcellulose | 1% |
| Kaolin | 82% |

The active ingredient is mixed and ground with the adjuvants, and the mixture is moistened with water. The mixture is extruded and then dried in a stream of air.

| Coated granules | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

| Suspension concentrate | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

| Flowable concentrate for seed treatment | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution | 0.5% |
| in water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### Preparation Examples

Using techniques described above and below, and also in WO 2000/046184, WO07031513, WO 2008/110313 WO 2010/086118 and WO 2008/110278, together with further techniques generally known to the person skilled in the art, compounds of formula (I) may be prepared.

### Example 1

### Preparation of N-ethyl-N′-(5-methoxy-2-methyl-4-phenoxy-phenyl)-N-methyl-formamidine

### Step 1. Preparation of 1-methoxy-4-methyl-5-nitro-2-phenoxy-benzene

A suspension of 1-chloro-2-methoxy-5-methyl-4-nitrobenzene (1.0 g, 4.96 mmol), phenol (0.56 g, 5.95 mmol) and K₂CO₃ (0.90 g, 6.45 mmol) in dry dimethyl formamide (10 mL) was warmed to 120° C. and stirred for 22 h at this temperature. The reaction was cooled to room temperature, diluted with ethyl acetate and washed with water (2×). The organic layer was washed with brine, dried over MgSO₄, solids were removed by filtration and volatiles were removed in vacuo. The residue was purified by flash chromatography on silica gel to afford the title compound as light yellow oil.

¹H NMR (400 MHz, CDCl₃): δ 7.73 (s, 1H), 7.35-7.45 (m, 2H), 7.14-7.24 (m, 1H), 6.99-7.08 (m, 2H), 6.69 (s, 1H), 3.94 (s, 3H), 2.49 (s, 3H).

### Step 2. Preparation of 5-methoxy-2-methyl-4-phenoxy-aniline

1-methoxy-4-methyl-5-nitro-2-phenoxy-benzene (0.99 g, 3.8 mmol) was dissolved in acetic acid (8 mL), warmed to 80° C. and iron dust (0.85 g, 15.3 mmol) was added portion wise at this temperature. Upon completed addition, the reaction mixture was stirred for an additional 60 min at 80° C. The mixture was cooled to room temperature, diluted with dichloromethane and filtrated through a pad of Celite. The filtrate was concentrated in vacuo to dark oil which was dissolved in ethyl acetate and washed with aq. NaHCO3. The organic layer was washed with brine, dried over MgSO₄, solids were removed by filtration and volatiles were removed in vacuo. The residue was purified by flash chromatography on silica gel to afford the title compound as light brown solid.

¹H NMR (400 MHz, CDCl₃): δ 7.18-7.30 (m, 2H), 6.97 (t, 1H), 6.82-6.92 (m, 2H), 6.75 (s, 1H), 6.37 (s, 1H), 3.74 (s, 3H), 2.08 (s, 3H).

### Step 3. Preparation of N-ethyl-N′-(5-methoxy-2-methyl-4-phenoxy-phenyl)-N-methyl-formamidine

A solution of 5-methoxy-2-methyl-4-phenoxy-aniline (0.55 g, 2.40 mmol) and N-(dimethoxymethyl)-N-methyl-ethanamine (0.64 g, 4.80 mmol) in toluene (5 mL) was treated with p-toluene sulfonic acid (1 small crystal) and warmed to 60° C. After stirring for 18 h at 60° C., the reaction was cooled to room termperature, diluted with ethyl acetate and washed with aq. NaHCO3. The organic layer was washed with brine, dried over MgSO₄, solids were removed by filtration and volatiles were removed in vacuo. The residue was purified by flash chromatography on silica gel to afford the title compound as light yellow oil.

¹H NMR (400 MHz, CDCl₃): δ 7.45 (br s, 1H), 7.16-7.32 (m, 2H), 6.94-7.06 (m, 1H), 6.91-6.89 (m, 2H), 6.81 (s, 1H), 6.44 (s, 1H), 3.78 (s, 3H), 3.38 (br s, 2H), 3.01 (s, 3H), 2.16 (s, 3H), 1.22 (t, 3H).

### Example 2

### Preparation of N′-[4-(4,5-dichlorothiazol-2-yl)oxy-5-methoxy-2-methyl-phenyl]-N-ethyl-N-methyl-formamidine

### Step 1. Preparation of 2-methoxy-5-methyl-4-nitro-phenol

A solution of 1,2-dimethoxy-4-methyl-5-nitro-benzene (0.50 g, 2.54 mmol) and lithium chloride (0.32 g, 7.61 mmol) in dimethyl formamide (10 mL) was heated to 170° C. using a microwave reactor and kept at this temperature for 3 h. The resulting dark solution was cooled to room temperature and poured into aq. NH4Cl solution. The mixture was extracted with tertbutyl methyl ether and the organic layer was washed with water to remove dimethyl formamide. The organic layer was extracted with aq. NaOH solution (1 M) and water. This combined aqueous extractes were then acified with conc. HCl to pH 1 and extracted with CH2Cl2. The dichloromethane layer was dried over MgSO4, filtrated and concentrated in vacuo to afford the title compound as yellow solid.

¹H NMR (400 MHz, CDCl₃): δ 7.68 (s, 1H), 6.83 (s, 1H), 6.01 (s, 1H), 3.96 (s, 3H), 2.58 (s, 3H).

### Step 2. Preparation of 4,5-dichloro-2-(2-methoxy-5-methyl-4-nitro-phenoxy)thiazole

Sodium hydride (60% in paraffin oil, 0.052 g, 1.4 mmol) was added to a solution of 2-methoxy-5-methyl-4-nitro-phenol (0.19 g, 1.0 mmol) in dry dimethyl formamide (1 mL) at room temperature. The resulting red solution was stirred for 45 min at room temperature before 2,4,5-trichlorothiazole (0.19 g, 1.0 mmol) was added. The resulting solution was warmed to 70° C. and stirred for 5 d at this temperature. The reaction was cooled to room temperature and diluted with ice water. The precipitated brown solid was collected on a glass filter, washed with cold water and dried in vacuo to afford the title compound.

¹H NMR (400 MHz, CDCl₃): δ 7.69 (s, 1H), 7.22 (s, 1H), 3.89 (s, 3H), 2.57 (s, 3H).

### Step 3. Preparation of 4-(4,5-dichlorothiazol-2-yl)oxy-5-methoxy-2-methyl-aniline

A solution of 4,5-dichloro-2-(2-methoxy-5-methyl-4-nitro-phenoxy)thiazole (0.28 g, 0.82 mmol) in ethanol (6 mL) was treated with NH4Cl (0.09 g, 1.64 mmol), water (1.5 mL) and iron dust (0.18 g, 3.28 mmol) at room temperature. The resulting mixture was warmed to 85° C. and stirred for 1 h at this temperature. After cooling to room temperature, the reaction was diluted with aq. NaHCO3 and extracted with ethyl acetate. The organic layer was washed with brine, dried over MgSO4, filtrated and concentrated in vacuo. The residue was purified by flash chromatography on silica gel to afford the title compound as light brown oil.

¹H NMR (400 MHz, CDCl₃): δ 6.88 (s, 1H), 6.32 (s, 1H), 3.76 (s, 3H), 3.69 (br s, 2H), 2.09 (s, 3H).

### Step 4. Preparation of N′[4-(4,5-dichlorothiazol-2-yl)oxy-5-methoxy-2-methyl-phenyl]-N-ethyl-N-methyl-formamidine

A solution of 4-(4,5-dichlorothiazol-2-yl)oxy-5-methoxy-2-methyl-aniline (0.32 g, 1.04 mmol) and N-(dimethoxymethyl)-N-methyl-ethanamine (0.42 g, 3.15 mmol) in toluene (3 mL) was treated with p-toluene sulfonic acid (1 small crystal) and warmed to 70° C. After stirring for 18 h at 70° C., the reaction was cooled to room termperature, diluted with ethyl acetate and washed with aq. NaHCO3. The organic layer was washed with brine, dried over MgSO₄, solids were removed by filtration and volatiles were removed in vacuo. The residue was purified by flash chromatography on silica gel to afford the title compound as light brown oil.

¹H NMR (400 MHz, CDCl₃): δ 7.43 (br s, 1H), 6.96 (s, 1H), 6.41 (s, 1H), 3.79 (s, 3H), 3.22-3.56 (m, 2H), 3.01 (s, 3H), 2.18 (s, 3H), 1.23 (t, 3H).

Table E: Physical data of compounds of formula (I) from Tables 1-32

The compounds of formula (I) were prepared using techniques described above and/or common synthetic techniques generally known to the person skilled in the art, as well as those described in WO 2000/046184, WO07031513, WO 2008/110313 WO 2010/086118 and WO 2008/110278.

TABLE E

| Compound No. | Melting point (° C.) | LC/MS |
|---|---|---|
| 1.009 | 59-60 | |
| 24.009 | | Rt = 0.82 min; MS: m/z 374 [M + 1]⁺ |
| 25.009 | | Rt = 0.61 min; MS: m/z 401 [M + 1]⁺ |
| 2.009 | | Rt = 0.63 min; MS: m/z 317 [M + 1]⁺ |
| 3.009 | | Rt = 0.82 min; MS: m/z 333 [M + 1]⁺ |
| 14.009 | | Rt = 0.65 min; MS: m/z 379 [M + 1]⁺ |
| 19.009 | | Rt = 0.71 min; MS: m/z 313 [M + 1]⁺ |

Throughout this description, temperatures are given in degrees Celsius and “m.p.” means melting point. LC/MS means Liquid Chromatography Mass Spectroscopy and the description of the apparatus and the method is as follows:

Spectra were recorded on a Mass Spectrometer from Waters equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 1.2-1.5 min 100% B; Flow (ml/min) 0.85

### Biological Examples

### Phakopsora pachyrhizi on Soybean, Preventive Treatment

The compound activity was tested under 1 day preventive conditions. Soybean plants with a fully enfolded first trifoliate leaf were sprayed with a track sprayer and 50 I/ha spray volume with the test compounds and rates as shown in the table below.

1 day after application leaf discs were cut from the first trifoliate leaf and placed in multiwell plates on water-agar. 5 leaf discs per treatment where infested with spores of a triazole- and strobilurine-tolerant soybean rust strain. The multiwell plates where sealed and placed in an incubator 48 h in darkness and 12 h light/dark cycle afterwards. Rust infestation on leaf discs was evaluated visually 10 days after application and average activity calculated in relation to disease severity on untreated check leaf discs.

### Phytotoxicity on Soybean, Preventive Treatment

Soybean plants with a fully enfolded first trifoliate leaf were sprayed with a track sprayer and 50 l/ha spray volume with the test compounds and rates as shown in the table below.

The plants were then transferred to a greenhouse at 22° C. and a 14 h day and 10 h night cycle. 5 plants per treatment were sprayed and evaluated. Phytotoxicity on plant leaves was evaluated visually 8 days after application and average phytotoxicity calculated.

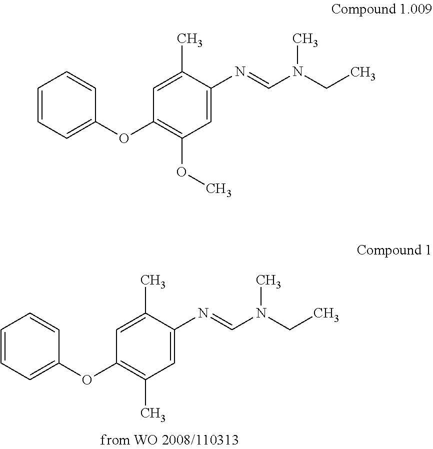 [SMILES: CCN(C)/C=N/c1cc(OC)c(Oc2ccccc2)cc1C ; CCN(C)/C=N/c1cc(C)c(Oc2ccccc2)cc1C]

| g | % soybean | % PHAKPA | g | % soybean | % PHAKPA |
|---|---|---|---|---|---|
| a.i./ha | phytotoxicity | activity | a.i./ha | phytotoxicity | activity |
| 32 | 5 | n.t. | 32 | 22 | n.t. |
| 16 | 3 | n.t. | 16 | 11 | n.t. |
| 8 | 5 | 100 | 8 | 9 | 100 |
| 4 | n.t. | 99 | 4 | n.t. | 97 |

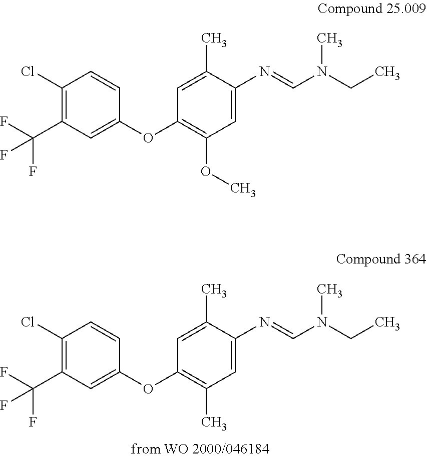 [SMILES: CCN(C)/C=N/c1cc(OC)c(Oc2ccc(Cl)c(C(F)(F)F)c2)cc1C ; CCN(C)/C=N/c1cc(C)c(Oc2ccc(Cl)c(C(F)(F)F)c2)cc1C]

| g | % soybean | % PHAKPA | g | % soybean | % PHAKPA |
|---|---|---|---|---|---|
| a.i./ha | phytotoxicity | activity | a.i./ha | phytotoxicity | activity |
| 32 | 3 | n.t. | 32 | 22 | n.t. |
| 16 | 4 | n.t. | 16 | 18 | n.t. |
| 8 | 2 | 99 | 8 | 17 | 97 |
| 4 | n.t. | 96 | 4 | n.t. | 87 |

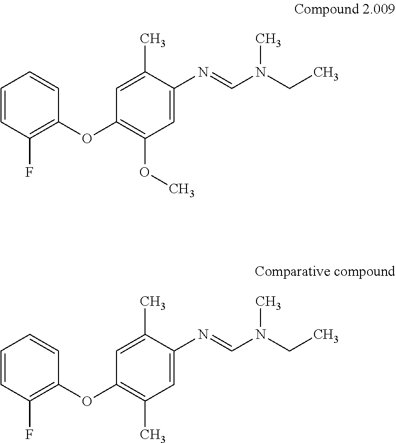 [SMILES: CCN(C)/C=N/c1cc(OC)c(Oc2ccccc2F)cc1C ; CCN(C)/C=N/c1cc(C)c(Oc2ccccc2F)cc1C]

| g | % soybean | % PHAKPA | g | % soybean | % PHAKPA |
|---|---|---|---|---|---|
| a.i./ha | phytotoxicity | activity | a.i./ha | phytotoxicity | activity |
| 32 | 8 | n.t. | 32 | 35 | n.t. |
| 16 | 5 | n.t. | 16 | 25 | n.t. |
| 8 | 4 | 96 | 8 | 18 | 95 |
| 4 | n.t. | 89 | 4 | n.t. | 93 |

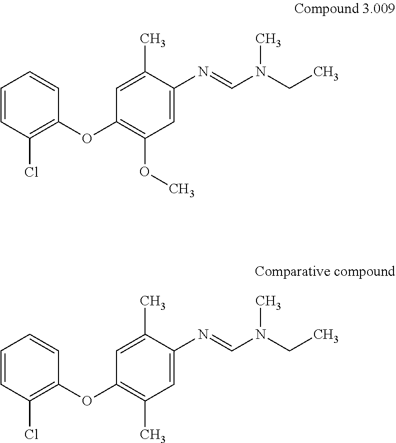 [SMILES: CCN(C)/C=N/c1cc(OC)c(Oc2ccccc2Cl)cc1C ; CCN(C)/C=N/c1cc(C)c(Oc2ccccc2Cl)cc1C]

| g | % soybean | % PHAKPA | g | % soybean | % PHAKPA |
|---|---|---|---|---|---|
| a.i./ha | phytotoxicity | activity | a.i./ha | phytotoxicity | activity |
| 32 | n.t. | n.t. | 32 | 35 | n.t. |
| 16 | 5 | n.t. | 16 | 32 | n.t. |
| 8 | 3 | 96 | 8 | 7 | 98 |
| 4 | n.t. | 95 | 4 | n.t. | 98 |

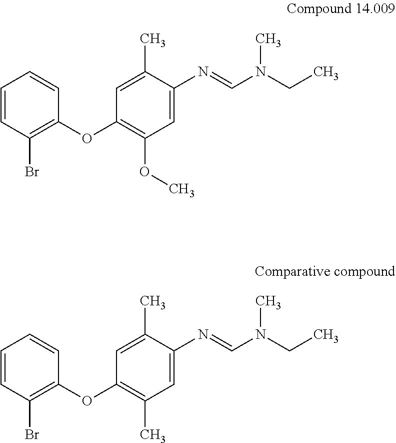 [SMILES: CCN(C)/C=N/c1cc(OC)c(Oc2ccccc2Br)cc1C ; CCN(C)/C=N/c1cc(C)c(Oc2ccccc2Br)cc1C]

| g | % soybean | % PHAKPA | g | % soybean | % PHAKPA |
|---|---|---|---|---|---|
| a.i./ha | phytotoxicity | activity | a.i./ha | phytotoxicity | activity |
| 32 | 5 | n.t. | 32 | 33 | n.t. |
| 16 | 4 | n.t. | 16 | 15 | n.t. |
| 8 | 2 | 93 | 8 | 7 | 96 |
| 4 | n.t. | 91 | 4 | n.t. | 97 |

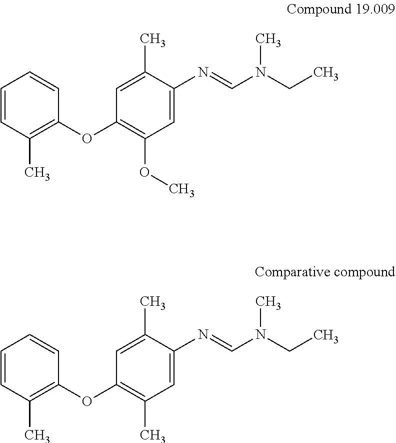 [SMILES: CCN(C)/C=N/c1cc(OC)c(Oc2ccccc2C)cc1C ; CCN(C)/C=N/c1cc(C)c(Oc2ccccc2C)cc1C]

| g | % soybean | % PHAKPA | g | % soybean | % PHAKPA |
|---|---|---|---|---|---|
| a.i./ha | phytotoxicity | activity | a.i./ha | phytotoxicity | activity |
| 32 | 11 | n.t. | 32 | 27 | n.t. |
| 16 | 5 | n.t. | 16 | 22 | n.t. |
| 8 | 5 | 98 | 8 | 10 | 95 |
| 4 | n.t. | 84 | 4 | n.t. | 97 |

What is claimed is:

1. A method of improving plant safety whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I) 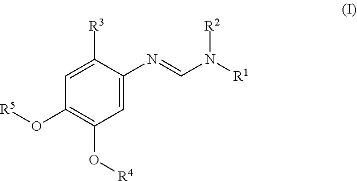
wherein,
R¹ and R² are each independently selected from C₁-C₄alkyl and C₃-C₈cycloalkyl; or
R¹ and R² together with the nitrogen atom to which they are attached form a three to six-membered saturated cyclic group which may optionally contain one oxygen or one sulphur atom;
R³ is C₁-C₄ alkyl or halogen; or
R³ is halomethyl (preferably CF₃ or CHF₂);
R⁴ is C₁-C₄alkyl or C₁-C₄haloalkyl;
R⁵ is aryl (optionally substituted with one to three R⁶ groups) or heteroaryl (optionally substituted with one to three R⁶ groups); and
each R⁶ is independently selected from halogen, cyano, hydroxyl, amino, nitro, C₁-C₄alkyl, C₁-C₄haloalkyl, C₃-C₆cycloalkyl, C₃-C₆halocycloalkyl, C₁-C₄alkoxy, C₁-C₄haloalkoxy, C₃-C₆cycloalkoxy, C₁-C₄alkylthio, C₁-C₄haloalkylthio, C₃-C₆cycloalkylthio, C₁-C₄alkylsulfinyl, C₁-C₄haloalkylsulfinyl, C₁-C₄alkylsulfonyl, C₁-C₄haloalkyl sulfonyl, C₁-C₄alkylcarbonyl, C₁-C₄alkoxycarbonyl, C₁-C₄alkylcarbonyloxy, C₂-C₆alkenyl, C₂-C₆haloalkenyl, C₂-C₆alkenyloxy, C₂-C₆haloalkenyloxy, C₂-C₆alkynyl, C₃-C₆cycloalkylC₂-C₆alkynyl, C₂-C₆alkynyloxy, aryl, aryl(C₁-C₄)alkyl, aryloxy, heteroaryl, heteroaryl(C₁-C₄)alkyl and heteroaryloxy; or a salt or an N-oxide thereof.

2. A method according to claim 1 wherein R¹ and R² are each independently C₁-C4 alkyl; R³ is C₁-C₃ alkyl; and R⁴ is C₁-C₄alkyl.

3. A method according to claim 1 wherein R⁵ is phenyl (optionally substituted with one to three R⁶ groups), pyridyl (optionally substituted with one to three R⁶ groups) or thiazolyl (optionally substituted with one to three R⁶ groups), and wherein each R⁶ is independently selected from halogen, cyano, C₁-C₄alkyl, C₁-C4haloalkyl, C₃-C₆cycloalkyl, C₁-C₄alkoxy, C₁-C₄haloalkoxy, C₃-C₆cycloalkoxy, C₁-C4alkylthio, C₁-C₄alkylsulfonyl, C₁-C₄haloalkylsulfonyl, C₁-C₄alkylcarbonyl, C₂-C₆alkenyl, C₂-C₆haloalkenyl, C₂-C₆alkynyl, phenyl, benzyl, phenoxy, pyridyl, pyridylmethyl and pyridyloxy.

4. A method according to claim 1 wherein R¹ and R² are each independently selected from methyl, ethyl and isopropyl; R³ is methyl, ethyl or isopropyl; and R⁴ is C₁-C₃alkyl.

5. A method according to claim 1 wherein R⁵ is phenyl (optionally substituted with one or two R⁶ groups) or thiazolyl (optionally substituted with one or two R⁶ groups), and wherein each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₁-C₄alkylthio, C₁-C₄alkylsulfonyl, C₁-C₄alkylcarbonyl, C₂-C₆alkynyl, phenyl, phenoxy and pyridyl.

6. A method according to claim 1 wherein R¹ and R² are each independently selected from methyl and ethyl; R³ is methyl or ethyl; R⁴ is methyl or ethyl; R⁵ is phenyl (optionally substituted with one or two R⁶ groups); and each R⁶ is independently selected from halogen, cyano, C₁-C₄alkyl, C₁-C4haloalkyl, C₁-C₄alkoxy, C₁-C₄haloalkoxy, C₂-C₆alkynyl and phenyl.

7. A compound of formula (IH) 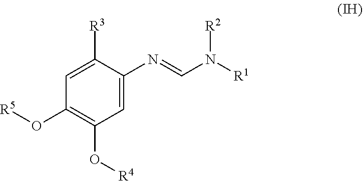
wherein
R¹ and R² are each independently selected from C₁-C₄alkyl and C₃-C₈cycloalkyl;
R³ is C₁-C₄ alkyl; or
R³ is fluoro, chloro, bromo, iodo or halomethyl (preferably chloro, bromo or CHF₂);
R⁴ is C₁-C₄alkyl or C₁-C₄haloalkyl;
R⁵ is aryl (optionally substituted with one to three R⁶ groups) or heteroaryl (optionally substituted with one to three R⁶ groups); and
each R⁶ is independently selected from halogen, cyano, hydroxyl, amino, nitro, C₁-C4alkyl, C₁-C₄haloalkyl, C₃-C₆cycloalkyl, C₃-C₆halocycloalkyl, C₁-C₄alkoxy, C₁-C4haloalkoxy, C₃-C₆cycloalkoxy, C₁-C₄alkylthio, C₁-C₄haloalkylthio, C₃-C6cycloalkylthio, C₁-C₄alkylsulfinyl, C₁-C₄haloalkylsulfinyl, C₁-C₄alkylsulfonyl, C₁-C₄haloalkylsulfonyl, C₁-C₄alkylcarbonyl, C₁-C₄alkoxycarbonyl, C₁-C₄alkylcarbonyloxy, C₂-C₆alkenyl, C₂-C₆haloalkenyl, C₂-C₆alkenyloxy, C₂-C₆haloalkenyloxy, C₂-C₆alkynyl, C₃-C₆cycloalkylC₂-C₆alkynyl, C₂-C₆alkynyloxy, aryl, aryl(C₁-C₄)alkyl, aryloxy, heteroaryl, heteroaryl(C₁-C₄)alkyl and heteroaryloxy; or a salt or an N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, 5-Chloro-3-(trifluoromethyl)phenyl, 4-Chloro-3-(isopropyl)phenyl, 4-Chloro-3-(tert-butyl)phenyl, or 5-cyclopropyl-1,3,4-thiadiazol-2-yl.

8. A compound according to claim 7, or a salt or an N-oxide thereof, wherein R¹ and R² are each independently C₁-C₄ alkyl; R³ is C₁-C₃ alkyl; and R⁴ is C₁-C₄alkyl.

9. A compound according to claim 7 wherein R¹ and R² are each independently C₁-C₄ alkyl; R³ is C₁-C₃ alkyl; R⁴ is C₁-C₄alky; R⁵ is phenyl (optionally substituted with one to three R⁶ groups), pyridyl (optionally substituted with one to three R⁶ groups) or thiazolyl (optionally substituted with one to three R⁶ groups); and each R⁶ is independently selected from halogen, cyano, C₁-C₄alkyl, C₁-C₄haloalkyl, C3-C6cycloalkyl, C₁-C₄alkoxy, C₁-C₄haloalkoxy, C₃-C₆cycloalkoxy, C₁-C₄alkylthio, C₁-C₄alkylsulfonyl, C₁-C₄haloalkylsulfonyl, C₁-C₄alkylcarbonyl, C₂-C₆alkenyl, C₂-C₆haloalkenyl, C₂-C₆alkynyl, phenyl, benzyl, phenoxy, pyridyl, pyridylmethyl and pyridyloxy; or a salt or N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, 5-Chloro-3-(trifluoromethyl)phenyl, 4-Chloro-3-(isopropyl)phenyl, or 4-Chloro-3-(tert-butyl)phenyl.

10. A compound according to claim 7 wherein R¹ and R² are each independently selected from methyl, ethyl and isopropyl; R³ is methyl, ethyl or isopropyl; R⁴ is C₁-C₃alkyl; R⁵ is phenyl (optionally substituted with one or two R⁶ groups) or thiazolyl (optionally substituted with one or two R⁶ groups); and each R⁶ is independently selected from halogen, cyano, C₁-C₄alkyl, C₁-C₄haloalkyl, C₃-C₆cycloalkyl, C₁-C₄alkoxy, C₁-C₄haloalkoxy, C₁-C₄alkylthio, C₁-C₄alkylsulfonyl, C₁-C₄alkylcarbonyl, C₂-C₆alkynyl, phenyl, phenoxy and pyridyl; or a salt or N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, 5-Chloro-3-(trifluoromethyl)phenyl, 4-Chloro-3-(isopropyl)phenyl, or 4-Chloro-3-(tert-butyl)phenyl.

11. A compound according to claim 7 wherein R¹ and R² are each independently selected from methyl and ethyl; R³ is methyl or ethyl; R⁴ is methyl or ethyl; R⁵ is phenyl (optionally substituted with one or two R⁶ groups); and each R⁶ is independently selected from halogen, cyano, C₁-C₄alkyl, C₁-C₄haloalkyl, C₁-C₄alkoxy, C₁-C₄haloalkoxy, C₂-C₆alkynyl and phenyl; or a salt or N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, 5-Chloro-3-(trifluoromethyl)phenyl, 4-Chloro-3-(isopropyl)phenyl, or 4-Chloro-3-(tert-butyl)phenyl.

12. A compound according to claim 7 wherein R¹ is methyl and R² is ethyl; R³ is methyl; R⁴ is methyl; and R⁵ is phenyl, which is optionally substituted by one or two sub stituents independently selected from trifluoromethyl and halogen (preferably fluoro or chloro); or a salt or N-oxide thereof, provided that when R¹ is methyl and R² is methyl or R¹ is methyl and R² is ethyl and R³ and R⁴ are both methyl then R⁵ is not 4-Chloro-3-(trifluoromethyl)phenyl, or 5 -Chloro-3-(trifluoromethyl)phenyl.

13. A compound according to claim 7 wherein the compound is: 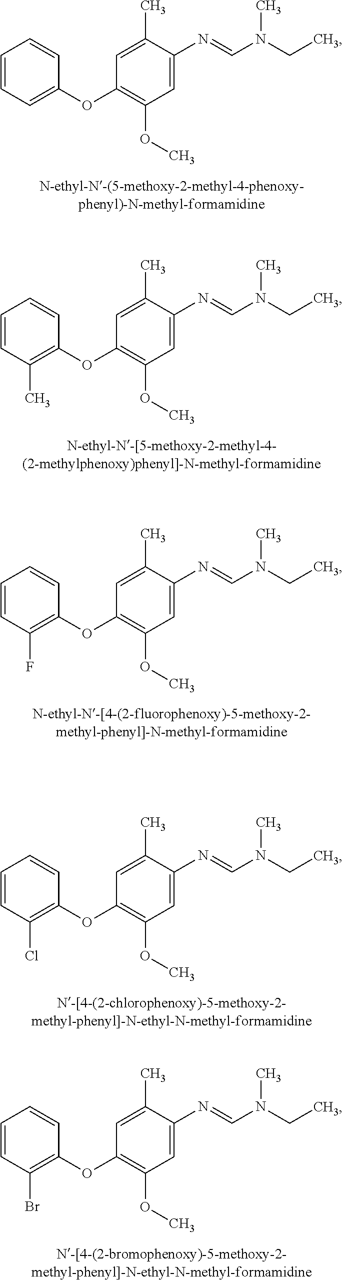 [SMILES: CCN(C)/C=N/c1cc(OC)c(Oc2ccccc2)cc1C ; CCN(C)/C=N/c1cc(OC)c(Oc2ccccc2C)cc1C ; CCN(C)/C=N/c1cc(OC)c(Oc2ccccc2F)cc1C ; CCN(C)/C=N/c1cc(OC)c(Oc2ccccc2Cl)cc1C ; CCN(C)/C=N/c1cc(OC)c(Oc2ccccc2Br)cc1C] 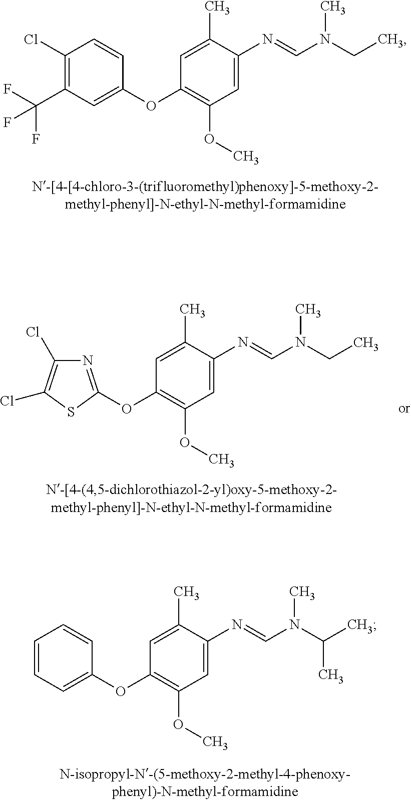 [SMILES: CCN(C)/C=N/c1cc(OC)c(Oc2ccc(Cl)c(C(F)(F)F)c2)cc1C ; CCN(C)/C=N/c1cc(OC)c(Oc2nc(Cl)c(Cl)s2)cc1C ; "or" ; COc1cc(/N=C/N(C)C(C)C)c(C)cc1Oc1ccccc1]
or a salt or an N-oxide thereof;
or wherein the compound is: 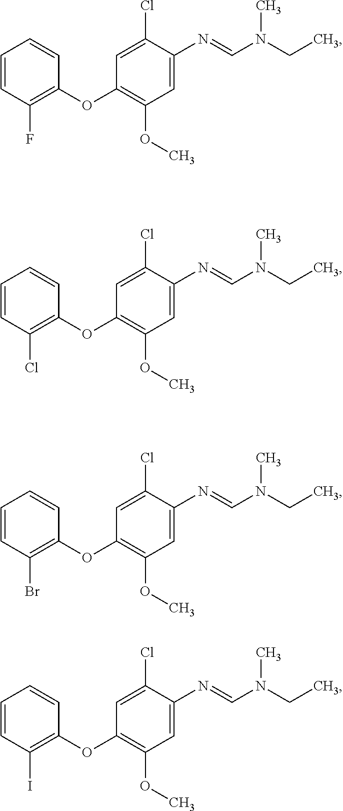 [SMILES: CCN(C)/C=N/c1cc(OC)c(Oc2ccccc2F)cc1Cl ; CCN(C)/C=N/c1cc(OC)c(Oc2ccccc2Cl)cc1Cl ; CCN(C)/C=N/c1cc(OC)c(Oc2ccccc2Br)cc1Cl ; CCN(C)/C=N/c1cc(OC)c(Oc2ccccc2I)cc1Cl] 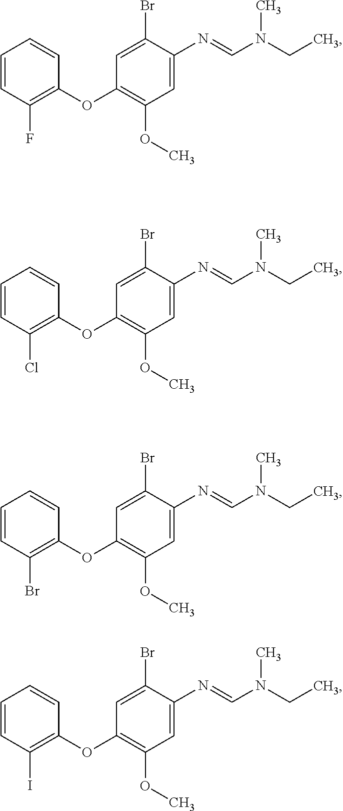 [SMILES: CCN(C)/C=N/c1cc(OC)c(Oc2ccccc2F)cc1Br ; CCN(C)/C=N/c1cc(OC)c(Oc2ccccc2Cl)cc1Br ; CCN(C)/C=N/c1cc(OC)c(Oc2ccccc2Br)cc1Br ; CCN(C)/C=N/c1cc(OC)c(Oc2ccccc2I)cc1Br] 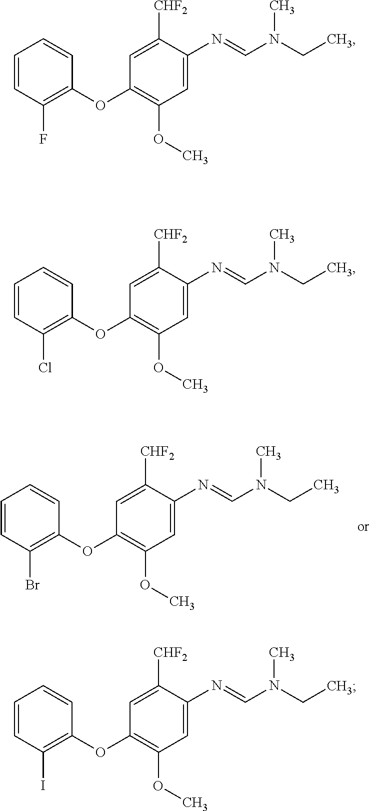 [SMILES: CCN(C)/C=N/c1cc(OC)c(Oc2ccccc2F)cc1C(F)F ; CCN(C)/C=N/c1cc(OC)c(Oc2ccccc2Cl)cc1C(F)F ; CCN(C)/C=N/c1cc(OC)c(Oc2ccccc2Br)cc1C(F)F ; "or" ; CCN(C)/C=N/c1cc(OC)c(Oc2ccccc2I)cc1C(F)F]
or a salt or an N-oxide thereof.

14. A composition comprising a fungicidally effective amount of a compound of formula (I) as defined in claim 7, wherein the composition optionally comprises one or more additional active ingredients and/or a diluent.

15. A method of reducing phytotoxicity whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), or a salt or an N-oxide thereof, as defined in claim 1.

16. A method of reducing plant necrosis whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), or a salt or an N-oxide thereof, as defined in claim 1.

17. A method of reducing phytotoxicity whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), or a salt or an N-oxide thereof, as defined in claim 7.

18. A method of reducing phytotoxicity whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), or a salt or an N-oxide thereof, as defined in claim 14.

19. A method of reducing plant necrosis whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), or a salt or an N-oxide thereof, as defined in claim 7.

20. A method of reducing plant necrosis whilst combating, preventing or controlling phytopathogenic diseases, which comprises applying to a phytopathogen, to the locus of a phytopathogen, or to a plant susceptible to attack by a phytopathogen, or to propagation material thereof, a fungicidally effective amount of a compound of formula (I), or a salt or an N-oxide thereof, as defined in claim 14.
